(19) United States
(12) Patent Application Publication
(10) Pub. No.: US 2008/0004325 A1
(43) Pub. Date: Jan. 3, 2008

(54) PTP1B inhibitors

(75) Inventors: Jinbo Lee, Andover, MA (US); Zhao-Kui Wan, Arlington, MA (US); Douglas P. Wilson, Ayer, MA (US); Eva Binnun, Boston, MA (US); David V. Erbe, Arlington, MA (US); Eddine Saiah, Brookline, MA (US)

Correspondence Address: FISH & RICHARDSON P.C., P.O BOX 1022, MINNEAPOLIS, MN, 55440-1022, US

(73) Assignee: Wyeth, Madison, NJ (US)

(21) Appl. No.: 11/824,007

(22) Filed: Jun. 29, 2007

Related U.S. Application Data
(60) Provisional application No. 60/817,837, filed on Jun. 29, 2006.

(51) Int. Cl. A61K 31/433; C07D 417/04

(52) U.S. Cl. 514/362; 548/135

(57) ABSTRACT

This invention relates to modulating (e.g., inhibiting) protein tyrosine phosphatase 1b (PTP1b).

### CROSS REFERENCE TO RELATED APPLICATIONS

This application claims the benefit of U.S. Provisional Application No. 60/817,837, filed on Jun. 29, 2006, which is incorporated herein by reference in its entirety.

### TECHNICAL FIELD

This invention relates to modulating (e.g., inhibiting) protein tyrosine phosphatase 1b (PTP1b).

### BACKGROUND

Diabetes is generally characterized by relatively high levels of plasma glucose (hyperglycemia) in the fasting state. Type 2 diabetics can often develop insulin resistance, in which the effect of insulin in stimulating glucose and lipid metabolism is diminished. Further, patients who have developed insulin resistance, but not type 2 diabetes, are also at risk of developing Syndrome X (metabolic syndrome). Syndrome X is characterized by insulin resistance, along with obesity (e.g., abdominal obesity), hyperinsulinemia, high blood pressure, relatively low HDL and relatively high VLDL. Although current treatments for type 2 diabetes can result in reduced levels of blood sugar, side effects can include weight and edema.

Protein tyrosine phosphatase 1b (PTP1b), a ˜50 kd intracelluar PTPase abundant in various human tissues, has been studied for its potential role as a negative regulator of insulin signaling. Some studies have shown that PTP1b is a negative regulator of insulin signaling. For example, mice deficient in PTP1b were healthy and showed increased insulin sensitivity and resistance to diet-induced obesity. These mice had lower glucose, insulin and triglyceride levels as well as improved insulin sensitivity as measured by glucose and insulin tolerance tests. PTP1b has also been implicated in attenuation of leptin receptor signaling. PTP1b deficient mice were shown to be more sensitive to leptin, which may explain in part their resistance to weight gain when placed on a high fat diet. Thus, the main target tissues for PTP1b inhibition appear to be insulin action in muscle and liver, as well as leptin signaling in the brain, while the commercial diabetes drugs, the peroxisome proliferative activated receptor-gamma (PPAR-γ) agonist class of insulin sensitizers, target adipose tissue. Thus, inhibition of PTP1b provides a target for regulating a variety of metabolic responses important to obesity and type 2 diabetes.

### SUMMARY

In one aspect, this invention relates to compounds having formula (I):

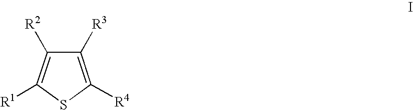

in which:

(a) one of R¹, R², R³, or R⁴ has formula (II):

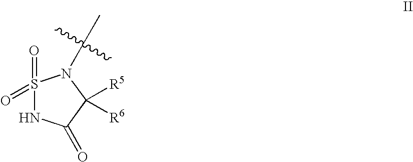

wherein R⁵ and R⁶ are each, independently, hydrogen, halo, or C₁-C₁₂ alkyl;

(b) one of R¹, R², R³, or R⁴ is C₆-C₁₆ aryl or heteroaryl including 5-16 atoms, each of which is optionally substituted with from 1-10 R^{a}; and

(c) two of R¹, R², R³, or R⁴ are each, independently:

(i) hydrogen; or

(ii) halo; NR^{b}R^{c}; nitro; azido; hydroxy; C₁-C₁₂ alkoxy or C₁-C₁₂ thioalkoxy, each of which is optionally substituted with 1-5 R^{d}; C₁-C₁₂ haloalkoxy; C₆-C₁₆ aryloxy, C₆-C₁₆ thioaryloxy, heteroaryloxy including 5-20 atoms, or thioheteroaryloxy including 5-20 atoms, each of which is optionally substituted with 1-5 R^{a}; C₂-C₁₂ alkenyloxy or C₂-C₁₂ alkynyloxy, each of which is optionally substituted with 1-5 R^{e}; C₃-C₁₆ cycloalkyloxy, C₃-C₁₆ cycloalkenyloxy, heterocyclyloxy including 3-16 atoms, heterocycloalkenyloxy including 3-16 atoms, C₇-C₂₀ aralkoxy, or heteroaralkoxy including 6-20 atoms, each of which is optionally substituted with 1-5 R^{f}; mercapto; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}SOₙR^{j}; or —P(O)(OR^{b})(OR^{c}); or

(iii) C₁-C₁₂ alkyl or C₁-C₁₂ haloalkyl; each of which is optionally substituted with from 1-5 R^{d}; or

(iv) C₂-C₂₀ alkenyl or C₂-C₂₀ alkynyl, each of which is optionally substituted with from 1-10 R^{e};

R^{a} at each occurrence is, independently:

(i) halo; NR^{b}R^{c}; nitro; azido; hydroxy; C₁-C₂₀ alkoxy or C₁-C₂₀ haloalkoxy, each of which is optionally substituted with from 1-10 R^{d}; C₆-C₁₈ aryloxy or heteroaryloxy including 5-16 atoms, each of which is optionally substituted with from 1-10 R^{a} or R^{a′}; C₂-C₁₂ alkenyloxy or C₂-C₁₂ alkynyloxy, each of which is optionally substituted with 1-5 R^{e}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{f}; mercapto; C₁-C₂₀ thioalkoxy or C₁-C₂₀ thiohaloalkoxy, each of which is optionally substituted with from 1-10 R^{d}; C₆-C₁₈ thioaryloxy or thioheteroaryloxy including 5-16 atoms, each of which is optionally substituted with from 1-10 R^{a} or R^{a′}; C₂-C₁₂ thioalkenyloxy or C₂-C₁₂ thioalkynyloxy, each of which is optionally substituted with 1-5 R^{e}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{f}; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c—; —NR}^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or

(ii) C₁-C₂₀ alkyl or C₁-C₂₀ haloalkyl, each of which is optionally substituted with from 1-10 R^{d}; or

(iii) C₂-C₂₀ alkenyl or C₂-C₂₀ alkynyl, each of which is optionally substituted with from 1-10 R^{e}; or

(iv) C₃-C₂₀ cycloalkyl C₃-C₂₀ cycloalkenyl, heterocyclyl including 3-20 atoms, or heterocycloalkenyl including 3-20 atoms, C₇-C₂₀ aralkyl or heteroaralkyl including 6-20 atoms, each of which is optionally substituted with from 1-10 R^{f}; or

(v) C₆-C₁₈ aryl or heteroaryl including 5-16 atoms, each of which is optionally substituted with from 1-10 R^{a} or R^{a′};

R^{a′}at each occurrence is, independently, halo; NR^{b}R^{c}; nitro; azido; hydroxy; C₁-C₂₀ alkyl, C₁-C₂₀ haloalkyl, C₂-C₂₀ alkenyl; C₂-C₂₀ alkynyl; C₃-C₂₀ cycloalkyl; C₃-C₂₀ cycloalkenyl, heterocyclyl including 3-20 atoms; heterocycloalkenyl including 3-20 atoms; C₇-C₂₀ aralkyl; heteroaralkyl including 6-20 atoms; C₁-C₂₀ alkoxy; C₁-C₂₀ haloalkoxy; C₆-C₁₈ aryloxy; heteroaryloxy; C₂-C₁₂ alkenyloxy; C₂-C₁₂ alkynyloxy; C₇-C₂₀ aralkoxy; heteroaralkoxy including 6-20 atoms; C₃-C₁₆ cycloalkoxy; C₃-C₂₀ cycloalkenyloxy; heterocyclyloxy including 3-20 atoms; heterocycloalkenyloxy including 3-20 atoms; mercapto; C₁-C₂₀ thioalkoxy; C₁-C₂₀ thiohaloalkoxy; C₆-C₁₈ thioaryloxy; thioheteroaryloxy including 5-16 atoms; C₂-C₁₂ thioalkenyloxy; C₂-C₁₂ thioalkynyloxy; C₇-C₂₀ thioaralkoxy, thioheteroaralkoxy including 6-20 atoms, C₃-C₁₆ thiocycloalkoxy C₃-C₂₀ thiocycloalkenyloxy, thioheterocyclyloxy including 3-20 atoms, or thioheterocycloalkenyloxy including 3-20 atoms, each of which is optionally substituted with from 1-10 R^{f}; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c});

each of R^{b}, R^{c}, R^{g}, R^{h}, and Rⁱ, at each occurrence is, independently:

(i) hydrogen; or

(ii) C₁-C₂₀ alkyl or C₁-C₂₀ haloalkyl, each of which is optionally substituted with from 1-10 R^{d}; or

(iii) C₂-C₂₀ alkenyl or C₂-C₂₀ alkynyl, each of which is optionally substituted with from 1-10 R^{e}; or

(iv) C₃-C₂₀ cycloalkyl, C₃-C₂₀ cycloalkenyl, heterocyclyl including 3-20 atoms, or heterocycloalkenyl including 3-20 atoms, C₇-C₂₀ aralkyl, or heteroaralkyl including 6-20 atoms, each of which is optionally substituted with from 1-10 R^{f}; or

(v) C₆-C₁₈ aryl or heteroaryl including 5-16 atoms, each of which is optionally substituted with from 1-10 R^{a}; or

(vi) —C(O)R^{g}, —C(O)OR^{g}; or —S(O)ₙR^{j};

R^{d} at each occurrence is, independently:

(i) NR^{b}R^{c}; nitro; azido; hydroxy; oxo; thioxo; ═NRⁱ; C₁-C₂₀ alkoxy; C₁-C₂₀ haloalkoxy; C₆-C₁₈ aryloxy or heteroaryloxy including 5-16 atoms, each of which is optionally substituted with from 1-10 R^{a}; C₂-C₁₂ alkenyloxy or C₂-C₁₂ alkynyloxy, each of which is optionally substituted with 1-5 R^{e}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{f}; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{a}; C₂-C₁₂ thioalkenyloxy or C₂-C₁₂ thioalkynyloxy, each of which is optionally substituted with 1-5 R^{e}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{f}; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or

(ii) C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{f};

R^{e} at each occurrence is, independently:

(i) halo; NR^{g}R^{h}; nitro; azido; hydroxy; oxo; thioxo; ═NRⁱ; C₁-C₂₀ alkoxy or C₁-C₂₀ haloalkoxy, each of which is optionally substituted with from 1-10 R^{d}; C₆-C₁₈ aryloxy or heteroaryloxy including 5-16 atoms, each of which is optionally substituted with from 1-10 R^{a}; C₂-C₁₂ alkenyloxy or C₂-C₁₂ alkynyloxy, each of which is optionally substituted with 1-5 R^{e}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{f}; mercapto; C₁-C₂₀ thioalkoxy or C₁-C₂₀ thiohaloalkoxy, each of which is optionally substituted with from 1-10 R^{d}; C₆-C₁₈ thioaryloxy or thioheteroaryloxy including 5-16 atoms, each of which is optionally substituted with from 1-10 R^{a}; C₂-C₁₂ thioalkenyloxy or C₂-C₁₂ thioalkynyloxy, each of which is optionally substituted with 1-5 R^{e}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{f}; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or

(ii) C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{f}; or

(iii) C₆-C₁₈ aryl or heteroaryl including 5-16 atoms, each of which is optionally substituted with from 1-10 R^{a};

R^{f} at each occurrence is, independently:

(i) halo; NR^{b}R^{c}; nitro; azido; hydroxy; oxo; thioxo; ═NRⁱ; C₁-C₂₀ alkoxy or C₁-C₂₀ haloalkoxy, each of which is optionally substituted with from 1-10 R^{d}; C₆-C₁₈ aryloxy or heteroaryloxy including 5-16 atoms, each of which is optionally substituted with from 1-10 R^{a} or R^{a′}; C₂-C₁₂ alkenyloxy or C₂-C₁₂ alkynyloxy, each of which is optionally substituted with 1-5 R^{e}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{f}; mercapto; C₁-C₂₀ thioalkoxy or C₁-C₂₀ thiohaloalkoxy, each of which is optionally substituted with from 1-10 R^{d}; C₆-C₁₈ thioaryloxy or thioheteroaryloxy including 5-16 atoms, each of which is optionally substituted with from 1-10 R^{a} or R^{a′}; C₂-C₁₂ thioalkenyloxy or C₂-C₁₂ thioalkynyloxy, each of which is optionally substituted with 1-5 R^{e}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{f}; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or

(ii) C₁-C₂₀ alkyl or C₁-C₂₀ haloalkyl, each of which is optionally substituted with from 1-10 R^{d}; or

(iii) C₂-C₂₀ alkenyl or C₂-C₂₀ alkynyl, each of which is optionally substituted with from 1-10 R^{e}; or

(iv) C₆-C₁₈ aryl or heteroaryl including 5-16 atoms, each of which is optionally substituted with from 1-10 R^{a};

R^{j} is R^{g}, OR^{g}, or NR^{b}R^{c}; and n is 0, 1 or 2; or a salt (e.g., a pharmaceutically acceptable salt) thereof.

In another aspect, this invention relates to compounds of formula (IV):

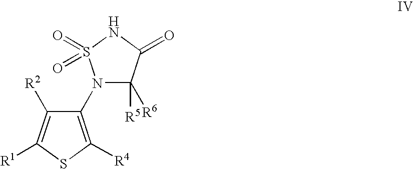

in which:

(b) one of R¹, R², or R⁴ (e.g., R¹) is C₆-C₁₆ aryl or heteroaryl including 5-16 atoms, each of which is optionally substituted with from 1-10 R^{a}; and

(c) two of R¹, R², or R⁴ (e.g., R² and R⁴) are each, independently:

(i) hydrogen; or (ii) halo; NR^{b}R^{c}; nitro; azido; hydroxy; C₁-C₁₂ alkoxy or C₁-C₁₂ thioalkoxy, each of which is optionally substituted with 1-5 R^{d}; C₁-C₁₂ haloalkoxy; C₆-C₁₆ aryloxy, C₆-C₁₆ thioaryloxy, heteroaryloxy including 5-20 atoms, or thioheteroaryloxy including 5-20 atoms, each of which is optionally substituted with 1-5 R^{a}; C₂-C₁₂ alkenyloxy or C₂-C₁₂ alkynyloxy, each of which is optionally substituted with 1-5 R^{e}; C₃-C₁₆ cycloalkyloxy, C₃-C₁₆ cycloalkenyloxy, heterocyclyloxy including 3-16 atoms, heterocycloalkenyloxy including 3-16 atoms, C₇-C₂₀ aralkoxy, or heteroaralkoxy including 6-20 atoms, each of which is optionally substituted with 1-5 R^{f}; mercapto; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or

(iii) C₁-C₁₂ alkyl or C₁-C₁₂ haloalkyl; each of which is optionally substituted with from 1-5 R^{d}; or

(iv) C₂-C₂₀ alkenyl or C₂-C₂₀ alkynyl, each of which is optionally substituted with from 1-10 R^{e}; and

R^{a} R^{a′}, R^{b}, R^{c}, R^{d}, R^{e}, R^{f}, R^{g}, R^{h}, Rⁱ, R^{j}, R⁵, and R⁶ can be as defined for formula (I).

In a further aspect, this invention relates to compounds of formula (IV):

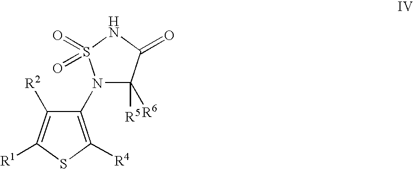

in which:

R¹is C₆-C₁₆ aryl or heteroaryl including 5-16 atoms, each of which is optionally substituted with from 1-10 R^{a};

R² and R⁴ are each, independently:

(i) hydrogen; or

(ii) halo; NR^{b}R^{c}; nitro; azido; hydroxy; C₁-C₁₂ alkoxy or C₁-C₁₂ thioalkoxy, each of which is optionally substituted with 1-5 R^{d}; C₁-C₁₂ haloalkoxy; C₆-C₁₆ aryloxy, C₆-C₁₆ thioaryloxy, heteroaryloxy including 5-20 atoms, or thioheteroaryloxy including 5-20 atoms, each of which is optionally substituted with 1-5 R^{a}; C₂-C₁₂ alkenyloxy or C₂-C₁₂ alkynyloxy, each of which is optionally substituted with 1-5 R^{e}; C₃-C₁₆ cycloalkyloxy, C₃-C₁₆ cycloalkenyloxy, heterocyclyloxy including 3-16 atoms, heterocycloalkenyloxy including 3-16 atoms, C₇-C₂₀ aralkoxy, or heteroaralkoxy including 6-20 atoms, each of which is optionally substituted with 1-5 R^{f}; mercapto; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or

(iii) C₁-C₁₂ alkyl or C₁-C₁₂ haloalkyl; each of which is optionally substituted with from 1-5 R^{d}; or

(iv) C₂-C₂₀ alkenyl or C₂-C₂₀ alkynyl, each of which is optionally substituted with from 1-10 R^{e}; and

R^{a}, R^{a′}, R^{b}, R^{c}, R^{d}, R^{e}, R^{f}, R^{g}, R^{h}, RⁱR^{j}, R⁵, and R⁶ can be as defined for formula (I).

In a further aspect, this invention relates to compounds of formula (V):

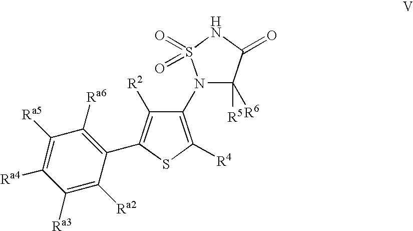

in which R² and R⁴ are as defined for formula (IV); and

R^{a2}, R^{a3}, R^{a4}, R^{a5}, and R^{a6} are each, independently:

(i) halo; NR^{b}R^{c} (e.g., NH₂, monosubstituted or disubstituted amino in which one or both, respectively, of R^{b} and R^{c} is other than hydrogen); nitro; hydroxy; C₁-C₁₂ (e.g., C₁-C₆) alkoxy or C₁-C₁₂ (e.g., C₁-C₆) haloalkoxy (e.g., OCF₃), each of which is optionally substituted with from 1-10 R^{d}; C₆-C₁₀ aryloxy or heteroaryloxy including 5-10 atoms, each of which is optionally substituted with from 1-5 R^{a} or R^{a′}; C₂-C₁₂ (e.g., C₂-C₆) alkenyloxy or C₂-C₁₂ (e.g., C₂-C₆) alkynyloxy, each of which is optionally substituted with 1-5 R^{e}; C₇-C₁₂ aralkoxy, heteroaralkoxy including 6-12 atoms, C₃-C₈ cycloalkoxy, C₃-C₈ cycloalkenyloxy, heterocyclyloxy including 3-8 atoms, or heterocycloalkenyloxy including 3-8 atoms, each of which is optionally substituted with from 1-10 R^{f}; mercapto; C₁-C₁₂ (e.g., C₁-C₆) thioalkoxy or C₁-C₁₂ (e.g., C₁-C₆) thiohaloalkoxy, each of which is optionally substituted with from 1-5 R^{d}; C₆-C₁₀ thioaryloxy or thioheteroaryloxy including 5-10 atoms, each of which is optionally substituted with from 1-5 R^{a} or R^{a′}; C₂-C₁₂ (e.g., C₂-C₆) thioalkenyloxy or C₂-C₁₂ (e.g., C₂-C₆) thioalkynyloxy, each of which is optionally substituted with 1-5 R^{e}; C₇-C₁₂ thioaralkoxy, thioheteroaralkoxy including 6-12 atoms, C₃-C₈ thiocycloalkoxy, C₃-C₈ thiocycloalkenyloxy, thioheterocyclyloxy including 3-8 atoms, or thioheterocycloalkenyloxy including 3-8 atoms, each of which is optionally substituted with from 1-10 R^{f}; cyano; —C(O)R^{g}, —C(O)OR^{g}; —OC(O)R^{g}; —C(O)SR^{g}; —C(O)NR^{b}R^{c}; NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; or —NR^{h}S(O)ₙR^{j}; or

(ii) C₁-C₁₂ (e.g., C₁-C₆) alkyl or C₁-C₁₂ (e.g., C₁-C₆) haloalkyl, each of which is optionally substituted with from 1-5 R^{d}; or

(iii) C₂-C₁₂ (e.g., C₂-C₆) alkenyl or C₂-C₁₂ (e.g., C₂-C₆) alkynyl, each of which is optionally substituted with from 1-5 R^{e}; or

(iv) C₇-C₂₀ aralkyl, optionally substituted with from 1-10 R^{f}; or

(v) hydrogen; and

R^{a}, R^{a′}, R^{b}, R^{c}, R^{d}, R^{e}, R^{f}, R^{g}, R^{h}, Rⁱ, R^{j}, R⁵, and R⁶ can be as defined anywhere herein.

In one aspect, this invention relates to any of the specific compounds delineated herein (including pharmaceutically acceptable salts thereof).

In one aspect, this invention features a pharmaceutical composition, which includes a compound of formula (I) (including any subgenera or specific compounds thereof) or a salt (e.g., a pharmaceutically acceptable salt) or a prodrug thereof and a pharmaceutically acceptable adjuvant, carrier or diluent. In some embodiments, the composition can include an effective amount of the compound or the salt thereof. In some embodiments, the composition can further include an additional therapeutic agent.

In one aspect, this invention relates to methods for treating diabetes (e.g., type 2 diabetes), which includes administering to a subject in need thereof an effective amount of a compound formula (I) (including any subgenera or specific compounds thereof) or a pharmaceutically acceptable salt thereof.

In one aspect, this invention relates to methods for treating obesity, which includes administering to a subject in need thereof an effective amount of a compound formula (I) (including any subgenera or specific compounds thereof) or a pharmaceutically acceptable salt thereof.

In one aspect, this invention relates to methods for increasing insulin sensitivity, which includes administering to a subject in need thereof (e.g., a type 2 diabetic) an effective amount of a compound formula (I) (including any subgenera or specific compounds thereof) or a pharmaceutically acceptable salt thereof.

In one aspect, this invention relates to methods for treating metabolic disorders, which includes administering to a subject in need thereof an effective amount of a compound formula (I) (including any subgenera or specific compounds thereof) or a pharmaceutically acceptable salt thereof.

The invention also relates generally to inhibiting PTP1b with the compounds described herein. In some embodiments, the methods can include, e.g., contacting a PTP1b in a sample (e.g., a tissue) with a compound of formula (I) (including any subgenera or specific compounds thereof). In other embodiments, the methods can include administering a compound of formula (I) (including any subgenera or specific compounds thereof) to a subject (e.g., a mammal, e.g., a human, e.g., a type 2 diabetic, e.g., an animal model for any of the diseases or disorders described herein). Accordingly, in yet another aspect, this invention includes methods of screening for compounds that inhibit PTP1b.

In some embodiments, the subject can be a subject in need thereof (e.g., a subject identified as being in need of such treatment). Identifying a subject in need of such treatment can be in the judgment of a subject or a health care professional and can be subjective (e.g. opinion) or objective (e.g. measurable by a test or diagnostic method). In some embodiments, the subject can be a mammal. In certain embodiments, the subject is a human.

In a further aspect, this invention also relates to methods of making compounds described herein. Alternatively, the method includes taking any one of the intermediate compounds described herein and reacting it with one or more chemical reagents in one or more steps to produce a compound described herein.

In one aspect, this invention relates to a packaged product. The packaged product includes a container, one of the aforementioned compounds in the container, and a legend (e.g., a label or an insert) associated with the container and indicating administration of the compound for treatment and control of diseases or disorders mediated by PTP1b, e.g., type 2 diabetes, obesity, metabolic disorders.

Embodiments can include one or more of the following features.

One of R¹, R², R³, or R⁴ can be C₆-C₁₀ aryl, optionally substituted with 1-3 R^{a}. R^{a} at each occurrence can be, independently:

(i) halo; NR^{b}R^{c}; nitro; hydroxy; C₁-C₁₂ alkoxy or C₁-C₁₂ haloalkoxy, each of which is optionally substituted with from 1-10 R^{d}; C₆-C₁₀ aryloxy or heteroaryloxy including 5-10 atoms, each of which is optionally substituted with from 1-5 R^{a} or R^{a}; C₂-C₁₂ alkenyloxy or C₂-C₁₂ alkynyloxy, each of which is optionally substituted with 1-5 R^{e}; C₇-C₁₂ aralkoxy, heteroaralkoxy including 6-12 atoms, C₃-C₈ cycloalkoxy, C₃-C₈ cycloalkenyloxy, heterocyclyloxy including 3-8 atoms, or heterocycloalkenyloxy including 3-8 atoms, each of which is optionally substituted with from 1-10 R^{f}; mercapto; C₁-C₁₂ thioalkoxy or C₁-C₁₂ thiohaloalkoxy, each of which is optionally substituted with from 1-5 R^{d}; C₆-C₁₈ thioaryloxy or thioheteroaryloxy including 5-10 atoms, each of which is optionally substituted with from 1-5 R^{a} or R^{a} ; C₂-C₁₂ thioalkenyloxy or C₂-C₁₂ thioalkynyloxy, each of which is optionally substituted with 1-5 R^{e}; C₇-C₁₂ thioaralkoxy, thioheteroaralkoxy including 6-12 atoms, C₃-C₈ thiocycloalkoxy, C₃-C₈ thiocycloalkenyloxy, thioheterocyclyloxy including 3-8 atoms, or thioheterocycloalkenyloxy including 3-8 atoms, each of which is optionally substituted with from 1-10 R^{f}; cyano; —C(O)R^{g}, —C(O)OR^{g}; —OC(O)R^{g}; —C(O)SR^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; or —NR^{h}S(O)ₙR^{j}; or

(ii) C₁-C₁₂ alkyl or C₁-C₁₂ haloalkyl, each of which is optionally substituted with from 1-5 R^{d}; or

(iii) C₂-C₁₂ alkenyl or C₂-C₁₂ alkynyl, each of which is optionally substituted with from 1-5 R^{e}; or

(iv) C₇-C₂₀ aralkyl, optionally substituted with from 1-10 R^{f}.

Two of R¹, R², R³, or R⁴ can each be, independently, hydrogen; halo; cyano, —C(O)OR^{g}; —C(O)NR^{b}R^{c}; or C₁-C₁₂ alkyl or C₁-C₁₂ haloalkyl, each of which is optionally substituted with from 1-5 R^{d}.

R⁵ and R⁶ can each be, independently, hydrogen, fluoro, or C₁-C₄ alkyl. For example, R⁵ and R⁶ can both be hydrogen; or one of R⁵ and R⁶ can be hydrogen, and the other can be C₁-C₄ alkyl.

R¹can be C₆-C₁₆ aryl or heteroaryl including 5-16 atoms, each of which is optionally substituted with from 1-10 R^{a}. R¹ can be C₆-C₁₆ aryl, optionally substituted with from 1-10 R^{a}. R¹ can be C₆-C₁₀ aryl, optionally substituted with 1-3 R^{a}. For example, R¹ can be phenyl, or R¹ can have formula (III):

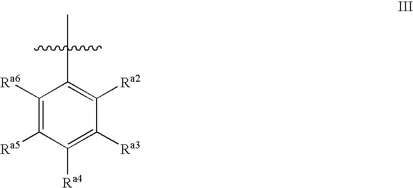

in which one of R^{a2}, R^{a3} , R^{a4} , R^{a5} and R^{a6} can be:

(i) halo; NR^{b}R^{c}; nitro; hydroxy; C₁-C₁₂ alkoxy or C₁-C₁₂ haloalkoxy, each of which is optionally substituted with from 1-10 R^{d}; C₆-C₁₀ aryloxy or heteroaryloxy including 5-10 atoms, each of which is optionally substituted with from 1-5 R^{a} or R^{a′}; C₂-C₁₂ alkenyloxy or C₂-C₁₂ alkynyloxy, each of which is optionally substituted with 1-5 R^{e}; C₇-C₁₂ aralkoxy, heteroaralkoxy including 6-12 atoms, C₃-C₈ cycloalkoxy, C₃-C₈ cycloalkenyloxy, heterocyclyloxy including 3-8 atoms, or heterocycloalkenyloxy including 3-8 atoms, each of which is optionally substituted with from 1-10 R^{f}; mercapto; C₁-C₁₂ thioalkoxy or C₁-C₁₂ thiohaloalkoxy, each of which is optionally substituted with from 1-5 R^{d}; C₆-C₁₈ thioaryloxy or thioheteroaryloxy including 5-10 atoms, each of which is optionally substituted with from 1-5 R^{a} or R^{a′}; C₂-C₁₂ thioalkenyloxy or C₂-C₁₂ thioalkynyloxy, each of which is optionally substituted with 1-5 R^{e}; C₇-C₁₂ thioaralkoxy, thioheteroaralkoxy including 6-12 atoms, C₃-C₈ thiocycloalkoxy, C₃-C₈ thiocycloalkenyloxy, thioheterocyclyloxy including 3-8 atoms, or thioheterocycloalkenyloxy including 3-8 atoms, each of which is optionally substituted with from 1-10 R^{f}; cyano; —C(O)R^{g}, —C(O)OR^{g}; —OC(O)R^{g}; —C(O)SR^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙRⁱ; or —NR^{h}S(O)ₙRⁱ; or

(ii) C₁-C₁₂ alkyl or C₁-C₁₂ haloalkyl, each of which is optionally substituted with from 1-5 R^{d}; or

(iii) C₂-C₁₂ alkenyl or C₂-C₁₂ alkynyl, each of which is optionally substituted with from 1-5 R^{e}; or

(iv) C₇-C₂₀ aralkyl, optionally substituted with from 1-10 R^{f}; and the others can be hydrogen.

For example, R^{a3} can be nitro.

As another example, R^{a3} can be NR^{b}R^{c}.

In some embodiments, one of R^{b} and R^{c} can be hydrogen, and the other can be:

(ii) C₁-C₂₀ alkyl or C₁-C₂₀ haloalkyl, each of which is optionally substituted with from 1-10 R^{d}; or

(iii) C₂-C₂₀ alkenyl or C₂-C₂₀ alkynyl, each of which is optionally substituted with from 1-10 R^{e}; or

(iv) C₃-C₂₀ cycloalkyl, C₃-C₂₀ cycloalkenyl, heterocyclyl including 3-20 atoms, or heterocycloalkenyl including 3-20 atoms, C₇-C₂₀ aralkyl, or heteroaralkyl including 6-20 atoms, each of which is optionally substituted with from 1-10 R^{f}; or

(v) C₆-C₁₈ aryl or heteroaryl including 5-16 atoms, each of which is optionally substituted with from 1-10 R^{a}; or

(vi) —S(O)ₙR^{j}, wherein R^{j} is NR^{b}R^{c}.

In certain embodiments, one of R^{b} and R^{c} can be hydrogen, and the other can be C₃-C₂₀ cycloalkyl, C₃-C₂₀ cycloalkenyl, heterocyclyl including 3-20 atoms, or heterocycloalkenyl including 3-20 atoms, C₇-C₂₀ aralkyl, or heteroaralkyl including 6-20 atoms.

In certain embodiments, one of R^{b} and R^{c} can be hydrogen, and the other can be C₃-C₁₀ cycloalkyl or heterocyclyl including 3-10 atoms, each of which is optionally substituted with from 1-10 R^{f}. For example, one of R^{b} and R^{c} can be hydrogen, and the other can be cyclohexyl, optionally substituted with from 1-5 R^{f}. As another example, one of R^{b} and R^{c} can be hydrogen, and the other can be 4-piperidyl, optionally substituted with from 1-2 R^{f}.

In some embodiments, R^{b} and R^{c} can each be, independently:

(ii) C₁-C₂₀ alkyl or C₁-C₂₀ haloalkyl, each of which is optionally substituted with from 1-10 R^{d}; or

(iii) C₂-C₂₀ alkenyl or C₂-C₂₀ alkynyl, each of which is optionally substituted with from 1-10 R^{e}; or

(iv) C₃-C₂₀ cycloalkyl, C₃-C₂₀ cycloalkenyl, heterocyclyl including 3-20 atoms, or heterocycloalkenyl including 3-20 atoms, C₇-C₂₀ aralkyl, or heteroaralkyl including 6-20 atoms, each of which is optionally substituted with from 1-10 R^{f}; or

(v) C₆-C₁₈ aryl or heteroaryl including 5-16 atoms, each of which is optionally substituted with from 1-10 R^{a}; or

(vi) —S(O)ₙR^{j}, wherein R^{j} is NR^{b}R^{c}.

In certain embodiments, one of R^{b} and R^{c} can be C₃-C₂₀ cycloalkyl, C₃-C₂₀ cycloalkenyl, heterocyclyl including 3-20 atoms, or heterocycloalkenyl including 3-20 atoms, C₇-C₂₀ aralkyl, or heteroaralkyl including 6-20 atoms, each of which is optionally substituted with from 1-10 R^{f}, and the other can be —S(O)ₙRⁱ, wherein R^{j} can be, for example, NR^{b}R^{c}.

In certain embodiments, one of R^{b} and R^{c} is C₃-C₂₀ cycloalkyl, optionally substituted with from 1-10 R^{f}, and the other can be —S(O)ₙR^{j}, wherein R^{j} can be, for example, NR^{b}R^{c}. For example, one of R^{b} and R^{c} can be cyclohexyl, optionally substituted with from 1-5 R^{f}, and the other can be —S(O)₂NH₂.

R³ can have formula (II).

R² and R⁴ can each be, independently:

(i) hydrogen; or

(ii) halo; NR^{b}R^{c}; nitro; azido; hydroxy; C₁-C₁₂ alkoxy or C₁-C₁₂ thioalkoxy, each of which is optionally substituted with 1-5 R^{d}; C₁-C₁₂ haloalkoxy; C₆-C₁₆ aryloxy, C₆-C₁₆ thioaryloxy, heteroaryloxy including 5-20 atoms, or thioheteroaryloxy including 5-20 atoms, each of which is optionally substituted with 1-5 R^{a}; C₂-C₁₂ alkenyloxy or C₂-C₁₂ alkynyloxy, each of which is optionally substituted with 1-5 R^{e}; C₃-C₁₆ cycloalkyloxy, C₃-C₁₆ cycloalkenyloxy, heterocyclyloxy including 3-16 atoms, heterocycloalkenyloxy including 3-16 atoms, C₇-C₂₀ aralkoxy, or heteroaralkoxy including 6-20 atoms, each of which is optionally substituted with 1-5 R^{f}; mercapto; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or

(iii) C₁-C₁₂ alkyl or C₁-C₁₂ haloalkyl; each of which is optionally substituted with from 1-5 R^{d}; or

(iv) C₂-C₂₀ alkenyl or C₂-C₂₀ alkynyl, each of which is optionally substituted with from 1-10 R^{e}.

In some embodiments, one of R² and R⁴ can be hydrogen, and the other can be:

(ii) halo; NR^{b}R^{c}; nitro; azido; hydroxy; C₁-C₁₂ alkoxy or C₁-C₁₂ thioalkoxy, each of which is optionally substituted with 1-5 R^{d}; C₁-C₁₂ haloalkoxy; C₆-C₁₆ aryloxy, C₆-C₁₆ thioaryloxy, heteroaryloxy including 5-20 atoms, or thioheteroaryloxy including 5-20 atoms, each of which is optionally substituted with 1-5 R^{a}; C₂-C₁₂ alkenyloxy or C₂-C₁₂ alkynyloxy, each of which is optionally substituted with 1-5 R^{e}; C₃-C₁₆ cycloalkyloxy, C₃-C₁₆ cycloalkenyloxy, heterocyclyloxy including 3-16 atoms, heterocycloalkenyloxy including 3-16 atoms, C₇-C₂₀ aralkoxy, or heteroaralkoxy including 6-20 atoms, each of which is optionally substituted with 1-5 R^{f}; mercapto; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NR¹)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or

(iii) C₁-C₁₂ alkyl or C₁-C₁₂ haloalkyl; each of which is optionally substituted with from 1-5 R^{d}; or

(iv) C₂-C₂₀ alkenyl or C₂-C₂₀ alkynyl, each of which is optionally substituted with from 1-10 R^{e}.

In certain embodiments, R² can be:

(ii) halo; NR^{b}R^{c}; nitro; azido; hydroxy; C₁-C₁₂ alkoxy or C₁-C₁₂ thioalkoxy, each of which is optionally substituted with 1-5 R^{d}; C₁-C₁₂ haloalkoxy; C₆-C₁₆ aryloxy, C₆-C₆ thioaryloxy, heteroaryloxy including 5-20 atoms, or thioheteroaryloxy including 5-20 atoms, each of which is optionally substituted with 1-5 R^{a}; C₂-C₁₂ alkenyloxy or C₂-C,₂ alkynyloxy, each of which is optionally substituted with 1-5 R^{e}; C₃-C₁₆ cycloalkyloxy, C₃-C₁₆ cycloalkenyloxy, heterocyclyloxy including 3-16 atoms, heterocycloalkenyloxy including 3-16 atoms, C₇-C₂₀ aralkoxy, or heteroaralkoxy including 6-20 atoms, each of which is optionally substituted with 1-5 R^{f}; mercapto; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NR¹)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or

(iii) C₁-C₁₂ alkyl or C₁-C₁₂ haloalkyl; each of which is optionally substituted with from 1-5 R^{d}; or

(iv) C₂-C₂₀ alkenyl or C₂-C₂₀ alkynyl, each of which is optionally substituted with from 1-10 R^{e}; and R⁴ can be hydrogen.

In some embodiments, R² and R⁴ can each be, independently, hydrogen; halo; cyano, —C(O)OR^{g}; —C(O)NR^{b}R^{c}; or C₁-C₁₂ alkyl or C₁-C₁₂ haloalkyl, each of which is optionally substituted with from 1-5 R^{d}.

In certain embodiments, one of R² and R⁴ can be hydrogen, and the other can be halo; cyano, —C(O)OR^{g}; —C(O)NR^{b}R^{c}; or C₁-C₁₂ alkyl or C₁-C₁₂ haloalkyl, each of which is optionally substituted with from 1-5 R^{d}.

In certain embodiments, R² can be halo; cyano; —C(O)OR^{g}; —C(O)NR^{b}R^{c}; or C₁-C₁₂ alkyl or C₁-C₁₂ haloalkyl, each of which is optionally substituted with from 1-5 R^{d}, and R⁴ can be hydrogen.

R² can be hydrogen; halo; C₁-C₄ alkyl; or C₁-C₄ haloalkyl.

R⁴ can be hydrogen; halo; cyano; —C(O)OR^{g}; —C(O)NR^{b}R^{c}; C₁-C₄ haloalkyl; or C₁-C₄ alkyl, optionally substituted with from 1-3 R^{d}.

For example, R² and R⁴ can both be hydrogen; or R² can be C₁-C₄ alkyl, and R⁴ can be hydrogen; or R² can be halo, and R⁴ can be hydrogen; or R² and R⁴ can both be halo.

The term “mammal” includes organisms, which include mice, rats, cows, sheep, pigs, rabbits, goats, and horses, monkeys, dogs, cats, and preferably humans.

“An effective amount” refers to an amount of a compound that confers a therapeutic effect (e.g., treats, controls, ameliorates, prevents, delays the onset of, or reduces the risk of developing a disease, disorder, or condition or symptoms thereof) on the treated subject. The therapeutic effect may be objective (i.e., measurable by some test or marker) or subjective (i.e., subject gives an indication of or feels an effect). An effective amount of the compound described above may range from about 0.01 mg/Kg to about 1000 mg/Kg, (e.g., from about 0.1 mg/Kg to about 100 mg/Kg, from about 1 mg/Kg to about 100 mg/Kg). Effective doses will also vary depending on route of administration, as well as the possibility of co-usage with other agents.

The term “halo” or “halogen” refers to any radical of fluorine, chlorine, bromine or iodine.

In general, and unless otherwise indicated, substituent (radical) prefix names are derived from the parent hydride by either (i) replacing the “ane” in the parent hydride with the suffixes “yl,” “diyl,” “triyl,” “tetrayl,” etc.; or (ii) replacing the “e” in the parent hydride with the suffixes “yl,” “diyl,” “triyl,” “tetrayl,” etc. (here the atom(s) with the free valence, when specified, is (are) given numbers as low as is consistent with any established numbering of the parent hydride). Accepted contracted names, e.g., adamantyl, naphthyl, anthryl, phenanthryl, furyl, pyridyl, isoquinolyl, quinolyl, and piperidyl, and trivial names, e.g., vinyl, allyl, phenyl, and thienyl are also used herein throughout. Conventional numbering/lettering systems are also adhered to for substituent numbering and the nomenclature of fused, bicyclic, tricyclic, polycyclic rings.

The term “alkyl” refers to a saturated hydrocarbon chain that may be a straight chain or branched chain, containing the indicated number of carbon atoms. For example, C₁-C₂₀ alkyl indicates that the group may have from 1 to 20 (inclusive) carbon atoms in it. Any atom can be substituted. Examples of alkyl groups include without limitation methyl, ethyl, and tert-butyl.

The term “cycloalkyl” refers to saturated monocyclic, bicyclic, tricyclic, or other polycyclic hydrocarbon groups. Any atom can be substituted, e.g., by one or more substituents. A ring carbon serves as the point of attachment of a cycloalkyl group to another moiety. Cycloalkyl groups can contain fused rings. Fused rings are rings that share a common carbon atom. Cycloalkyl moieties can include, e.g., cyclopropyl, cyclobutyl, cyclopentyl, cyclohexyl, cycloheptyl, adamantyl, and norbornyl (bicycle[2.2.1]heptyl).

The term “haloalkyl” refers to an alkyl group, in which at least one hydrogen atom is replaced by halo. In some embodiments, more than one hydrogen atom (2, 3, 4, 5, 6, 7, 8, 9, 10, 11, 12, 13, 14, 15, 16, 17, 18, 19, 20, 21, 22, 23, 24, 25, 26,etc. hydrogen atoms) on a alkyl group can be replaced by more than one halogen (e.g., 2, 3, 4, 5, 6, 7, 8, 9, 10, 11, 12, 13, 14, 15, 16, 17, 18, 19, 20, 21, 22, 23, 24, 25, 26, etc. halogen atoms). In these embodiments, the hydrogen atoms can each be replaced by the same halogen (e.g., fluoro) or the hydrogen atoms can be replaced by a combination of different halogens (e.g., fluoro and chloro). “Haloalkyl” and also include alkyl moieties in which all hydrogens have been replaced by halo (e.g., perhaloalkyl, such as trifluoromethyl).

The term “aralkyl” refers to an alkyl moiety in which an alkyl hydrogen atom is replaced by an aryl group. One of the carbons of the alkyl moiety serves as the point of attachment of the aralkyl group to another moiety. Aralkyl includes groups in which more than one hydrogen atom on an alkyl moiety has been replaced by an aryl group. Any ring or chain atom can be substituted e.g., by one or more substituents. Examples of “aralkyl” include without limitation benzyl, 2-phenylethyl, 3-phenylpropyl, benzhydryl (diphenylmethyl), and trityl (triphenylmethyl) groups.

The term “heteroaralkyl” refers to an alkyl moiety in which an alkyl hydrogen atom is replaced by a heteroaryl group. One of the carbons of the alkyl moiety serves as the point of attachment of the aralkyl group to another moiety. Heteroaralkyl includes groups in which more than one hydrogen atom on an alkyl moiety has been replaced by a heteroaryl group. Any ring or chain atom can be substituted e.g., by one or more substituents. Heteroaralkyl can include, for example, 2-pyridylethyl.

The term “alkenyl” refers to a straight or branched hydrocarbon chain containing 2-20 carbon atoms and having one or more double bonds. Any atom can be substituted, e.g., by one or more substituents. Alkenyl groups can include, e.g., allyl, 1-butenyl, 2-hexenyl and 3-octenyl groups. One of the double bond carbons can optionally be the point of attachment of the alkenyl substituent. The term “alkynyl” refers to a straight or branched hydrocarbon chain containing 2-20 carbon atoms and having one or more triple bonds. Any atom can be substituted, e.g., by one or more substituents. Alkynyl groups can include, e.g., ethynyl, propargyl, and 3-hexynyl. One of the triple bond carbons can optionally be the point of attachment of the alkynyl substituent.

The term “alkoxy” refers to an —O-alkyl radical. The term “mercapto” refers to an SH radical. The term “thioalkoxy” refers to an —S-alkyl radical. The terms “aryloxy” and “heteroaryloxy” refer to an —O-aryl radical and —O-heteroaryl radical, respectively. The terms “thioaryloxy” and “thioheteroaryloxy” refer to an —S-aryl radical and —S-heteroaryl radical, respectively. The terms “alkenyloxy” and “alkynyloxy” refer to —O-alkenyl and —O-alkynyl radicals, respectively. The terms “thioalkenyloxy” and “thioalkynyloxy” refer to —S-alkenyl and —S-alkynyl radicals, respectively. The terms “aralkoxy” and “heteroaralkoxy” refer to an —O-aralkyl radical and —O-heteroaralkyl radical, respectively. The terms “thioaralkoxy” and “thioheteroaralkoxy” refer to an —S-aralkyl radical and —S-heteroaralkyl radical, respectively. The term “cycloalkoxy” refers to an —O-cycloalkyl radical. The terms “cycloalkenyloxy” and “heterocycloalkenyloxy” refer to an —O-cycloalkenyl radical and —O-heterocycloalkenyl radical, respectively. The term “heterocyclyloxy” refers to an —O-heterocyclyl radical. The term “thiocycloalkoxy” refers to an —S-cycloalkyl radical. The terms “thiocycloalkenyloxy” and “thioheterocycloalkenyloxy” refer to an —S-cycloalkenyl radical and —S-heterocycloalkenyl radical, respectively. The term “thioheterocyclyloxy” refers to an —S-heterocyclyl radical.

The term “heterocyclyl” refers to a saturated monocyclic, bicyclic, tricyclic or other polycyclic ring system having 1-4 heteroatoms if monocyclic, 1-8 heteroatoms if bicyclic, or 1-10 heteroatoms if tricyclic, said heteroatoms selected from O, N, or S (e.g., carbon atoms and 1-4, 1-8, or 1-10 heteroatoms of N, O, or S if monocyclic, bicyclic, or tricyclic, respectively). The heteroatom or ring carbon is the point of attachment of the heterocyclyl substituent to another moiety. Any atom can be substituted, e.g., by one or more substituents. The heterocyclyl groups can contain fused rings. Fused rings are rings that share a common carbon atom. Heterocyclyl groups can include, e.g., tetrahydrofuryl, tetrahydropyranyl, piperidyl (piperidino), piperazinyl, morpholinyl (morpholino), pyrrolinyl, and pyrrolidinyl.

The term “cycloalkenyl” refers to partially unsaturated monocyclic, bicyclic, tricyclic, or other polycyclic hydrocarbon groups. A ring carbon (e.g., saturated or unsaturated) is the point of attachment of the cycloalkenyl substituent. Any atom can be substituted e.g., by one or more substituents. The cycloalkenyl groups can contain fused rings. Fused rings are rings that share a common carbon atom. Cycloalkenyl moieties can include, e.g., cyclohexenyl, cyclohexadienyl, or norbornenyl.

The term “heterocycloalkenyl” refers to partially unsaturated monocyclic, bicyclic, tricyclic, or other polycyclic hydrocarbon groups having 1-4 heteroatoms if monocyclic, 1-8 heteroatoms if bicyclic, or 1-10 heteroatoms if tricyclic, said heteroatoms selected from O, N, or S (e.g., carbon atoms and 1-4, 1-8, or 1-10 heteroatoms of N, O, or S if monocyclic, bicyclic, or tricyclic, respectively). A ring carbon (e.g., saturated or unsaturated) or heteroatom is the point of attachment of the heterocycloalkenyl substituent. Any atom can be substituted, e.g., by one or more substituents. The heterocycloalkenyl groups can contain fused rings. Fused rings are rings that share a common carbon atom. Heterocycloalkenyl groups can include, e.g., tetrahydropyridyl, and dihydropyranyl.

The term “aryl” refers to an aromatic monocyclic, bicyclic, or tricyclic hydrocarbon ring system, wherein any ring atom can be substituted, e.g., by one or more substituents. Aryl groups can contain fused rings. Fused rings are rings that share a common carbon atom. Aryl moieties can include, e.g., phenyl, naphthyl, anthracenyl, and pyrenyl.

The term “heteroaryl” refers to an aromatic monocyclic, bicyclic, tricyclic, or other polycyclic hydrocarbon groups having 1-4 heteroatoms if monocyclic, 1-8 heteroatoms if bicyclic, or 1-10 heteroatoms if tricyclic, said heteroatoms selected from O, N, or S (e.g., carbon atoms and 1-4, 1-8, or 1-10 heteroatoms of N, O, or S if monocyclic, bicyclic, or tricyclic, respectively). Any atom can be substituted, e.g., by one or more substituents. Heteroaryl groups can contain fused rings. Fused rings are rings that share a common carbon atom. Heteroaryl groups include pyridyl, thienyl, furyl (furanyl), imidazolyl, isoquinolyl, quinolyl and pyrrolyl.

The term “oxo” refers to an oxygen atom, which forms a carbonyl (C═O) when attached to carbon. The term “thioxo” refers to an oxygen atom, which forms a thiocarbonyl (C═S) when attached to carbon. Descriptors such as C(O), C(S), and C(NRi) refer to carbon atoms that are doubly bonded to an oxygen, sulfur, and nitrogen atom, respectively.

The term “substituent” refers to a group “substituted” on, e.g., an alkyl, cycloalkyl, alkenyl, alkynyl, aralkyl, heteroaralkyl, heterocyclyl, heterocycloalkenyl, cycloalkenyl, aryl, heteroaryl, arylcycloalkenyl, arylheterocyclyl, or arylheterocycloalkenyl group at any atom of that group. In one aspect, the substituent(s) (e.g., R^{a}) on a group are independently any one single, or any combination of two or more of the permissible atoms or groups of atoms delineated for that substituent. In another aspect, a substituent may itself be substituted with any one of the above substituents (e.g., R^{a′}).

In general, when a definition for a particular variable (e.g., R^{a} or R^{a3}) includes both hydrogen and non-hydrogen (halo, alkyl, aryl, etc.) possibilities, the term “substituent(s) other than hydrogen” refers collectively to the non-hydrogen possibilities for that particular variable.

In some embodiments, the compounds have a reduced likelihood (e.g., relative to PPAR-γ agonist diabetes drugs) of producing weight gain associated side effects when administered to a subject, e.g., a subject in need of treatment of type 2 diabetes.

The details of one or more embodiments of the invention are set forth in the accompanying drawings and the description below. Other features and advantages of the invention will be apparent from the description and from the claims.

### DETAILED DESCRIPTION

This invention relates to PTP1b inhibitor compounds, pharmaceutical compositions and related methods.

The PTP1b inhibitor compounds have the general formula (I) below:

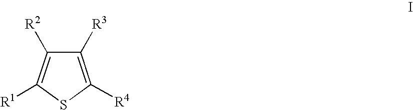

in which R¹, R², R³, R⁴, R⁵, R⁶, R^{a}, R^{a′}, R^{b}, R^{c}, R^{d}, R^{e}, R^{f}, R^{g}, R^{h}, RⁱR^{j}, and n can be as defined anywhere herein.

For ease of exposition, it is understood that any recitation of ranges (e.g., C₁-C₂₀, 1-3) or subranges of a particular range (e.g., C₁-C₄, C₂-C₆, 1-2) for any of R¹, R², R³, R⁴, R⁵, R⁶, R^{a}, R^{a′}, R^{b}, R^{c}, R^{d}, R^{e}, R^{f}, R^{g}, R^{h}, Rⁱ, R^{j}, and n expressly includes each of the individual values that fall within the recited range, including the upper and lower limits of the recited range. For example, the range C₁-C₄ alkyl is understood to mean C₁, C₂, C₃, or C₄ alkyl or the range 1-3 R^{a} is understood to mean 1, 2, or 3 R^{a}.

In all embodiments, one of R¹, R², R³, or R⁴ (e.g., R¹) is C₆-C₁₈ (e.g., C₆-C₁₄, C₆-C₁₀, or phenyl) aryl or heteroaryl including 5-20 (e.g., 5-16, 5-12, or 5-6) atoms, each of which is optionally substituted with from 1-10 (e.g., 1-5, 1-4, 1-3, 1-2, or 1) R^{a}.

In some embodiments, one of R¹, R², R³, or R⁴ (e.g., R¹) can be C₆-C₁₈ (e.g., C₆-C₁₄, C₆-C₁₀, or phenyl) aryl, optionally substituted with from 1-10 (e.g., 1-5, 1-4, 1-3, 1-2, or 1) R^{a}.

In certain embodiments, one of R¹, R², R³, or R⁴ (e.g., R¹) can be C₆-C₁₀ aryl, optionally substituted with 1-3 (e.g., 1-2 or 1) R^{a}.

In certain embodiments, one of R¹, R², R³, or R⁴ (e.g., R¹) can be unsubstituted phenyl.

In certain embodiments, one of R¹, R², R³, or R⁴ (e.g., R¹) can be a monosubstituted (1 R^{a}), disubstituted (2 R^{a}), trisubstituted (3 R^{a}), tetrasubstituted (4 R^{a}), or pentasubstituted (5 R^{a}) phenyl group.

In certain embodiments, R^{a} at each occurrence can be, independently:

(i) halo; NR^{b}R^{c}(e.g., NH₂, monosubstituted or disubstituted amino in which one or both, respectively, of R^{b} and R^{c} is other than hydrogen); nitro; hydroxy; C₁-C₁₂ (e.g., C₁-C₆) alkoxy or C₁-C₁₂ (e.g., C₁-C₆) haloalkoxy (e.g., OCF₃), each of which is optionally substituted with from 1-10 R^{d}; C₆-C₁₀ aryloxy or heteroaryloxy including 5-10 atoms, each of which is optionally substituted with from 1-5 R^{a} or R^{a′}; C₂-C₁₂ (e.g., C₂-C₆) alkenyloxy or C₂-C₁₂ (e.g., C₂-C₆) alkynyloxy, each of which is optionally substituted with 1-5 R^{e}; C₇-C₁₂ aralkoxy, heteroaralkoxy including 6-12 atoms, C₃-C₈ cycloalkoxy, C₃-C₈ cycloalkenyloxy, heterocyclyloxy including 3-8 atoms, or heterocycloalkenyloxy including 3-8 atoms, each of which is optionally substituted with from 1-10 R^{f}; mercapto; C₁-C₁₂ (e.g., C₁-C₆) thioalkoxy or C₁-C₁₂ (e.g., C₁-C₆) thiohaloalkoxy, each of which is optionally substituted with from 1-5 R^{d}; C₆-C₁₀ thioaryloxy or thioheteroaryloxy including 5-10 atoms, each of which is optionally substituted with from 1-5 R^{a} or R^{a′}; C₂-C₁₂ (e.g., C₂-C₆) thioalkenyloxy or C₂-C₁₂ (e.g., C₂-C₆) thioalkynyloxy, each of which is optionally substituted with 1-5 R^{e}; C₇-C₁₂ thioaralkoxy, thioheteroaralkoxy including 6-12 atoms, C₃-C₈ thiocycloalkoxy, C₃-C₈ thiocycloalkenyloxy, thioheterocyclyloxy including 3-8 atoms, or thioheterocycloalkenyloxy including 3-8 atoms, each of which is optionally substituted with from 1-10 R^{f}; cyano; —C(O)R^{g}, —C(O)OR^{g}; —OC(O)R^{g}; —C(O)SR^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; or —NR^{h}S(O)ₙR^{j}; or

(ii) C₁-C₁₂ (e.g., C₁-C₆) alkyl or C₁-C₁₂ (e.g., C₁-C₆) haloalkyl, each of which is optionally substituted with from 1-5 R^{d}; or

(iii) C₂-C₁₂ (e.g., C₂-C₆) alkenyl or C₂-C₁₂ (e.g., C₂-C₆) alkynyl, each of which is optionally substituted with from 1-5 R^{e}; or

(iv) C₇-C₂₀ aralkyl, optionally substituted with from 1-10 R^{f}.

In certain embodiments, when R^{a} is C₁-C₁₂ alkyl or C₁-C₁₂ haloalkyl that is optionally substituted with from 1-5 R^{d}, then R^{d} at each occurrence can be, independently:

(i) oxo; cyano; hydroxy; azido; (e.g., NH₂, monosubstituted or disubstituted amino in which one or both, respectively, of R^{b} and R^{c} is other than hydrogen); C₁-C,₂ (e.g., C₁-C₆) alkoxy or C₁-C₁₂ (e.g., C₁-C₆) haloalkoxy (e.g., OCF₃), each of which is optionally substituted with from 1-10 R^{d}; C₆-C₁₀ aryloxy or heteroaryloxy including 5-10 atoms, each of which is optionally substituted with from 1-5 R^{a} or R^{a′}; C₂-C₁₂ (e.g., C₂-C₆) alkenyloxy or C₂-C₁₂ (e.g., C₂-C₆) alkynyloxy, each of which is optionally substituted with 1-5 R^{e}; C₇-C₁₂ aralkoxy, heteroaralkoxy including 6-12 atoms, C₃-C₈ cycloalkoxy, C₃-C₈ cycloalkenyloxy, heterocyclyloxy including 3-8 atoms, or heterocycloalkenyloxy including 3-8 atoms, each of which is optionally substituted with from 1-10 R^{f}; mercapto; C₁-C,₂ (e.g., C₁-C₆) thioalkoxy or C₁-C₁₂ (e.g., C₁-C₆) thiohaloalkoxy, each of which is optionally substituted with from 1-5 R^{d}; C₆-C₁₀ thioaryloxy or thioheteroaryloxy including 5-10 atoms, each of which is optionally substituted with from 1-5 R^{a} or R^{a′}; C₂-C₁₂ (e.g., C₂-C₆) thioalkenyloxy or C₂-C₁₂ (e.g., C₂-C₆) thioalkynyloxy, each of which is optionally substituted with 1-5 R^{e}; C₇-C₁₂ thioaralkoxy, thioheteroaralkoxy including 6-12 atoms, C₃-C₈ thiocycloalkoxy, C₃-C₈ thiocycloalkenyloxy, thioheterocyclyloxy including 3-8 atoms, or thioheterocycloalkenyloxy including 3-8 atoms, each of which is optionally substituted with from 1-10 R^{f}; cyano; —C(O)R^{g}, —C(O)OR^{g}; —OC(O)R^{g}; —C(O)SR^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙRⁱ; or —NR^{h}S(O)ₙR^{j}; or

(ii) heterocyclyl including 3-10 atoms, optionally substituted with from 1-10 R^{f}.

In certain embodiments, when R^{a} is C₂-C₁₂ alkenyl or C₂-C₁₂ alkynyl that is optionally substituted with from 1-5 R^{e}, then R^{e} at each occurrence can be, independently:

(i) halo; oxo; cyano; hydroxy; azido; (e.g., NH₂, monosubstituted or disubstituted amino in which one or both, respectively, of R^{b} and R^{c} is other than hydrogen); C₁-C,₂ (e.g., C₁-C₆) alkoxy or C₁-C₁₂ (e.g., C₁-C₆) haloalkoxy (e.g., OCF₃), each of which is optionally substituted with from 1-10 R^{d}; C₆-C₁₀ aryloxy or heteroaryloxy including 5-10 atoms, each of which is optionally substituted with from 1-5 R^{a} or R^{a′}; C₂-C₁₂ (e.g., C₂-C₆) alkenyloxy or C₂-C₁₂ (e.g., C₂-C₆) alkynyloxy, each of which is optionally substituted with 1-5 R^{e}; C₇-C₁₂ aralkoxy, heteroaralkoxy including 6-12 atoms, C₃-C₈ cycloalkoxy, C₃-C₈ cycloalkenyloxy, heterocyclyloxy including 3-8 atoms, or heterocycloalkenyloxy including 3-8 atoms, each of which is optionally substituted with from 1-10 R^{f}; mercapto; C₁-C₁₂ (e.g., C₁-C₆) thioalkoxy or C₁-C₁₂ (e.g., C₁-C₆) thiohaloalkoxy, each of which is optionally substituted with from 1-5 R^{d}; C₆-C₁₀ thioaryloxy or thioheteroaryloxy including 5-10 atoms, each of which is optionally substituted with from 1-5 R^{a} or R^{a′}; C₂-C₁₂ (e.g., C₂-C₆) thioalkenyloxy or C₂-C₁₂ (e.g., C₂-C₆) thioalkynyloxy, each of which is optionally substituted with 1-5 R^{e}; C₇-C₁₂ thioaralkoxy, thioheteroaralkoxy including 6-12 atoms, C₃-C₈ thiocycloalkoxy, C₃-C₈ thiocycloalkenyloxy, thioheterocyclyloxy including 3-8 atoms, or thioheterocycloalkenyloxy including 3-8 atoms, each of which is optionally substituted with from 1-10 R^{f}; cyano; —C(O)R^{g}, —C(O)OR^{g}; —OC(O)R^{g}; —C(O)SR^{g}; C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; or —NR^{h}S(O)ₙR^{j}; or

(ii) heterocyclyl including 3-10 atoms, optionally substituted with from 1-10 R^{f}; or

(iii) C₆-C₁₀ aryl, optionally substituted with from 1-5 R^{a}.

In certain embodiments, when R^{a} is (or includes) a moiety that is substituted with one or more of R^{b}, R^{c}, R^{g}, and/or R^{h}, then each of R^{b}, R^{c}, R^{g}, and R^{h} (e.g., R^{b} and R^{c}; or R^{g}; or R^{h}) can be, independently of one another:

(i) hydrogen; or

(ii) C₁-C₁₆ alkyl or C₁-C₁₆ haloalkyl, each of which is optionally substituted with from 1-10 R^{d}; or

(iii) C₂-C₁₂ alkenyl or C₂-C₁₂ alkynyl, each of which is optionally substituted with from 1-10 R^{e}; or

(iv) C₃-C₈ cycloalkyl, heterocyclyl including 3-8 atoms, or C₇-C₂₀ aralkyl, each of which is optionally substituted with from 1-10 R^{f}; or

(v) C₆-Cl₀ aryl, optionally substituted with from 1-10 R^{a}; or

(vi) —S(O)ₙR^{j}.

In certain embodiments, when any of R^{b}, R^{c}, R^{g}, or R^{h} is (or includes) C₃-C₈ cycloalkyl (optionally substituted with from 1-10 R^{f}), heterocyclyl including 3-8 atoms (optionally substituted with from 1-10 R^{f}), or C₇-C₂₀ aralkyl (optionally substituted with from 1-10 R^{e}), or C₆-C₁₀ aryl that is optionally substituted with from 1-10 R^{a}, then R^{a} and R^{f} at each occurrence can be, independently:

(i) halo; NR^{b}R^{c}(e.g., NH₂, monosubstituted or disubstituted amino in which one or both, respectively, of R^{b} and R^{c} is other than hydrogen); nitro; hydroxy; C₁-C₁₂ (e.g., C₁-C₆) alkoxy or C₁-C₁₂ (e.g., C₁-C₆) haloalkoxy (e.g., OCF₃), each of which is optionally substituted with from 1-10 R^{d}; C₆-C₁₀ aryloxy or heteroaryloxy including 5-10 atoms, each of which is optionally substituted with from 1-5 R^{a} or R^{a′}; C₂-C₁₂ (e.g., C₂-C₆) alkenyloxy or C₂-C₁₂ (e.g., C₂-C₆) alkynyloxy, each of which is optionally substituted with 1-5 R^{e}; C₇-C₁₂ aralkoxy, heteroaralkoxy including 6-12 atoms, C₃-C₈ cycloalkoxy, C₃-C₈ cycloalkenyloxy, heterocyclyloxy including 3-8 atoms, or heterocycloalkenyloxy including 3-8 atoms, each of which is optionally substituted with from 1-10 R^{f}; mercapto; C₁-C,₂ (e.g., C₁-C₆) thioalkoxy or C₁-C₁₂ (e.g., C₁-C₆) thiohaloalkoxy, each of which is optionally substituted with from 1-5 R^{d}; C₆-C₁₀ thioaryloxy or thioheteroaryloxy including 5-10 atoms, each of which is optionally substituted with from 1-5 R^{a} or R^{a′}; C₂-C₁₂ (e.g., C₂-C₆) thioalkenyloxy or C₂-C₁₂ (e.g., C₂-C₆) thioalkynyloxy, each of which is optionally substituted with 1-5 R^{e}; C₇-C₁₂ thioaralkoxy, thioheteroaralkoxy including 6-12 atoms, C₃-C₈ thiocycloalkoxy, C₃-C₈ thiocycloalkenyloxy, thioheterocyclyloxy including 3-8 atoms, or thioheterocycloalkenyloxy including 3-8 atoms, each of which is optionally substituted with from 1-10 R^{f}; cyano; —C(O)R^{g}, —C(O)OR^{g}; —OC(O)R^{g}; —C(O)SR^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; or —NR^{h}S(O)ₙR^{j}; or

(ii) C₁-C₁₂ (e.g., C₁-C₆) alkyl or C₁-C₁₂ (e.g., C₁-C₆) haloalkyl, each of which is optionally substituted with from 1-5 R^{d} (in which R^{d} can be as defined anywhere herein); or

(iii) C₂-C₁₂ (e.g., C₂-C₆) alkenyl or C₂-C₁₂ (e.g., C₂-C₆) alkynyl, each of which is optionally substituted with from 1-5 R^{e} (in which R^{e} can be as defined anywhere herein).

In certain embodiments, when R^{b}, R^{c}, R^{g}, or R^{h} is (or includes) C₁-C₁₂ alkyl or C₁-C₁₂ haloalkyl that is optionally substituted with from 1-5 R^{d}, then R^{d} at each occurrence can be, independently:

(i) oxo; cyano; hydroxy; azido; (e.g., NH₂, monosubstituted or disubstituted amino in which one or both, respectively, of R^{b} and R^{c} is other than hydrogen); C₁-C,₂ (e.g., C₁-C₆) alkoxy or C₁-C₁₂ (e.g., C₁-C₆) haloalkoxy (e.g., OCF₃), each of which is optionally substituted with from 1-10 R^{d}; C₆-C₁₀ aryloxy or heteroaryloxy including 5-10 atoms, each of which is optionally substituted with from 1-5 R^{a} or R^{a′}; C₂-C₁₂ (e.g., C₂-C₆) alkenyloxy or C₂-C₁₂ (e.g., C₂-C₆) alkynyloxy, each of which is optionally substituted with 1-5 R^{e}; C₇-C₁₂ aralkoxy, heteroaralkoxy including 6-12 atoms, C₃-C₈ cycloalkoxy, C₃-C₈ cycloalkenyloxy, heterocyclyloxy including 3-8 atoms, or heterocycloalkenyloxy including 3-8 atoms, each of which is optionally substituted with from 1-10 R^{f}; mercapto; C₁-C,₂ (e.g., C₁-C₆) thioalkoxy or C₁-C₁₂ (e.g., C₁-C₆) thiohaloalkoxy, each of which is optionally substituted with from 1-5 R^{d}; C₆-C₁₀ thioaryloxy or thioheteroaryloxy including 5-10 atoms, each of which is optionally substituted with from 1-5 R^{a} or R^{a′}; C₂-C₁₂ (e.g., C₂-C₆) thioalkenyloxy or C₂-C₁₂ (e.g., C₂-C₆) thioalkynyloxy, each of which is optionally substituted with 1-5 R^{e}; C₇-C,₂ thioaralkoxy, thioheteroaralkoxy including 6-12 atoms, C₃-C₈ thiocycloalkoxy, C₃-C₈ thiocycloalkenyloxy, thioheterocyclyloxy including 3-8 atoms, or thioheterocycloalkenyloxy including 3-8 atoms, each of which is optionally substituted with from 1-10 R^{f}; cyano; —C(O)R^{g}, —C(O)OR^{g}; —OC(O)R^{g}; —C(O)SR^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; or —NR^{h}S(O)ₙR^{j}; or

(ii) C₃-C₈ cycloalkyl or heterocyclyl including 3-10 atoms, each of which is optionally substituted with from 1-10 R^{f} (in which R^{f} can be as defined anywhere herein).

In certain embodiments, when R^{b}, R^{c}, R^{g}, or R^{h} is (or includes) C₂-C₁₂ alkenyl or C₂-C₁₂ alkynyl that is optionally substituted with from 1-5 R^{e}, then R^{e} at each occurrence can be, independently:

(i) halo; oxo; cyano; hydroxy; azido; (e.g., NH₂, monosubstituted or disubstituted amino in which one or both, respectively, of R^{b} and R^{c} is other than hydrogen); C₁-C₁₂ (e.g., C₁-C₆) alkoxy or C₁-C₁₂ (e.g., C₁-C₆) haloalkoxy (e.g., OCF₃), each of which is optionally substituted with from 1-10 R^{d}; C₆-C₁₀ aryloxy or heteroaryloxy including 5-10 atoms, each of which is optionally substituted with from 1-5 R^{a} or R^{a′}; C₂-C₁₂ (e.g., C₂-C₆) alkenyloxy or C₂-C₁₂ (e.g., C₂-C₆) alkynyloxy, each of which is optionally substituted with 1-5 R^{e}; C₇-C₁₂ aralkoxy, heteroaralkoxy including 6-12 atoms, C₃-C₈ cycloalkoxy, C₃-C₈ cycloalkenyloxy, heterocyclyloxy including 3-8 atoms, or heterocycloalkenyloxy including 3-8 atoms, each of which is optionally substituted with from 1-10 R^{f}; mercapto; C₁-C₁₂ (e.g., C₁-C₆) thioalkoxy or C₁-C₁₂ (e.g., C₁-C₆) thiohaloalkoxy, each of which is optionally substituted with from 1-5 R^{d}; C₆-C₁₀ thioaryloxy or thioheteroaryloxy including 5-10 atoms, each of which is optionally substituted with from 1-5 R^{a} or R^{a′}; C₂-C₁₂ (e.g., C₂-C₆) thioalkenyloxy or C₂-C₁₂ (e.g., C₂-C₆) thioalkynyloxy, each of which is optionally substituted with 1-5 R^{e}; C₇-C₁₂ thioaralkoxy, thioheteroaralkoxy including 6-12 atoms, C₃-C₈ thiocycloalkoxy, C₃-C₈ thiocycloalkenyloxy, thioheterocyclyloxy including 3-8 atoms, or thioheterocycloalkenyloxy including 3-8 atoms, each of which is optionally substituted with from 1-10 R^{f}; cyano; —C(O)R^{g}, —C(O)OR^{g}; —OC(O)R^{g}; —C(O)SR^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; or —NR^{h}S(O)ₙR^{j}; or

(ii) C₃-C₈ cycloalkyl or heterocyclyl including 3-10 atoms, each of which is optionally substituted with from 1-10 R^{f} (in which R^{f} can be as defined anywhere herein) or

(iii) C₆-C₁₀ aryl, optionally substituted with from 1-5 R^{a} (in which R^{a} can be as defined anywhere herein).

A preferred subset of R^{b}, R^{c}, R^{g}, and R^{h} moieties includes C₇-C₂₀ aralkyl, optionally substituted with from 1-10 R^{f}; C₁-C₆ alkyl or C₁-C₆ haloalkyl that is substituted with a C₃-C₈ cycloalkyl or a heterocyclyl including 3-10 (e.g., 5-8) atoms, each of which is optionally substituted with from 1-10 R^{f} (the alkyl chain can also itself be further substituted with from 1-3 R^{d}); or C₂-C₁₂ alkenyl or C₂-C₁₂ alkynyl that is substituted with a C₃-C₈ cycloalkyl (optionally substituted with 1-10 R^{f}), a heterocyclyl including 3-10 (e.g., 5-8) atoms (optionally substituted with 1-10 R^{f}), or a C₆-C₁₀ aryl, optionally substituted with from 1-5 R^{a} (the alkenyl and alkynyl chains can also themselves be further substituted with from 1-3 R^{e}).

In certain embodiments, R^{a} can be other than phenyl or phenyl substituted with one or more of the following: C₁-C₆ alkyl; heterocyclyl including 3-10 atoms (e.g., heterocyclyl including 5-6 atoms); heteroaryl including 3-10 atoms (e.g., heteroaryl including 5-6 atoms); C₆-C₁₀ aryl (e.g., phenyl); C₆-C₁₀ aryloxy (e.g., phenoxy); or C₇-C₂₀ aralkyl (e.g., —CH(Ph)—CH(Ph)); and/or R^{a} can be other than NH₂ or an NR^{b}R^{c} moiety in which one of R^{b} and R^{c} is hydrogen, and the other is —C(O)R^{g}, —C(O)OR^{g}; or —S(O)ₙR^{j}.

In certain embodiments, one of R¹, R², R³, or R⁴ (e.g., R¹) can be a phenyl group having formula (III):

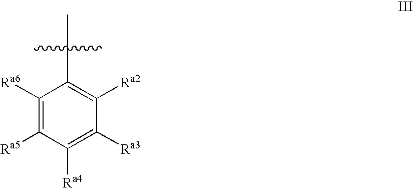

in which one, two, three, four, or five (e.g., one or two) of R^{a2}, R^{a3}, R^{a4}, R^{a5}, and R^{a6} can be, independently of one another:

(i) halo; NR^{b}R^{c}(e.g., NH₂, monosubstituted or disubstituted amino in which one or both, respectively, of R^{b} and R^{c} is other than hydrogen); nitro; hydroxy; C₁-C₁₂ (e.g., C₁-C₆) alkoxy or C₁-C₁₂ (e.g., C₁-C₆) haloalkoxy (e.g., OCF₃), each of which is optionally substituted with from 1-10 R^{d}; C₆-C₁₀ aryloxy or heteroaryloxy including 5-10 atoms, each of which is optionally substituted with from 1-5 R^{a} or R^{a′}; C₂-C₁₂ (e.g., C₂-C₆) alkenyloxy or C₂-C₁₂ (e.g., C₂-C₆) alkynyloxy, each of which is optionally substituted with 1-5 R^{e}; C₇-C₁₂ aralkoxy, heteroaralkoxy including 6-12 atoms, C₃-C₈ cycloalkoxy, C₃-C₈ cycloalkenyloxy, heterocyclyloxy including 3-8 atoms, or heterocycloalkenyloxy including 3-8 atoms, each of which is optionally substituted with from 1-10 R^{f}; mercapto; C₁-C,₂ (e.g., C₁-C₆) thioalkoxy or C₁-C₁₂ (e.g., C₁-C₆) thiohaloalkoxy, each of which is optionally substituted with from 1-5 R^{d}; C₆-C₁₀ thioaryloxy or thioheteroaryloxy including 5-10 atoms, each of which is optionally substituted with from 1-5 R^{a} or R^{a′}; C₂-C₁₂ (e.g., C₂-C₆) thioalkenyloxy or C₂-C₁₂ (e.g., C₂-C₆) thioalkynyloxy, each of which is optionally substituted with 1-5 R^{e}; C₇-C₁₂ thioaralkoxy, thioheteroaralkoxy including 6-12 atoms, C₃-C₈ thiocycloalkoxy, C₃-C₈ thiocycloalkenyloxy, thioheterocyclyloxy including 3-8 atoms, or thioheterocycloalkenyloxy including 3-8 atoms, each of which is optionally substituted with from 1-10 R^{f}; cyano; —C(O)R^{g}, —C(O)OR^{g}; —OC(O)R^{g}; —C(O)SR^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; or —NR^{h}S(O)ₙR^{j}; or

(ii) C₁-C₁₂ (e.g., C₁-C₆) alkyl or C₁-C₁₂ (e.g., C₁-C₆) haloalkyl, each of which is optionally substituted with from 1-5 R^{d}; or

(iii) C₂-C₁₂ (e.g., C₂-C₆) alkenyl or C₂-C₁₂ (e.g., C₂-C₆) alkynyl, each of which is optionally substituted with from 1-5 R^{e}; or

(iv) C₇-C₂₀ aralkyl, optionally substituted with from 1-10 R^{f};

and the other(s) can be hydrogen (except for embodiments in which all five of R^{a2}, R^{a3}, R^{a4}, R^{a5}, and R^{a6} are each a substituent other than hydrogen selected independently from (i)-(iv) above).

In general, variables R^{a2}, R^{a3}, R^{a4}, R^{a5}, and R^{a6} can include any one or more of the structural features or preferences described herein for variable R^{a}.

In certain embodiments, R^{a2}, R^{a3}, R^{a4}, R^{a5}, and R^{a6} can each be hydrogen.

In certain embodiments, the phenyl group of formula (III) can be a monosubstituted (e.g., an ortho, meta, or para monosubstituted) phenyl group (i.e., only one of R^{a2}, R^{a3}, R^{a4}, R^{a5}, and R^{a6} (e.g., R^{a3}) is a substituent other than hydrogen, and the other four are each hydrogen).

In certain embodiments, R^{a3} can be:

(i) halo; NR^{b}R^{c} (e.g., NH₂, monosubstituted or disubstituted amino in which one or both, respectively, of R^{b} and R_{c} is other than hydrogen); nitro; hydroxy; C₁-C₁₂ (e.g., C₁-C₆) alkoxy or C₁-C₁₂ (e.g., C₁-C₆) haloalkoxy (e.g., OCF₃), each of which is optionally substituted with from 1-10 R^{d}; C₆-C₁₀ aryloxy or heteroaryloxy including 5-10 atoms, each of which is optionally substituted with from 1-5 R^{a} or R^{a′}; C₂-C₁₂ (e.g., C₂-C₆) alkenyloxy or C₂-C₁₂ (e.g., C₂-C₆) alkynyloxy, each of which is optionally substituted with 1-5 R^{e}; C₇-C₁₂ aralkoxy, heteroaralkoxy including 6-12 atoms, C₃-C₈ cycloalkoxy, C₃-C₈ cycloalkenyloxy, heterocyclyloxy including 3-8 atoms, or heterocycloalkenyloxy including 3-8 atoms, each of which is optionally substituted with from 1-10 R^{f}; mercapto; C₁-C₁₂ (e.g., C₁-C₆) thioalkoxy or C₁-C₁₂ (e.g., C₁-C₆) thiohaloalkoxy, each of which is optionally substituted with from 1-5 R^{d}; C₆-C₁₀ thioaryloxy or thioheteroaryloxy including 5-10 atoms, each of which is optionally substituted with from 1-5 R^{a} or R^{a′}; C₂-C₁₂ (e.g., C₂-C₆) thioalkenyloxy or C₂-C₁₂ (e.g., C₂-C₆) thioalkynyloxy, each of which is optionally substituted with 1-5 R^{e}; C₇-C₁₂ thioaralkoxy, thioheteroaralkoxy including 6-12 atoms, C₃-C₈ thiocycloalkoxy, C₃-C₈ thiocycloalkenyloxy, thioheterocyclyloxy including 3-8 atoms, or thioheterocycloalkenyloxy including 3-8 atoms, each of which is optionally substituted with from 1-10 R^{f}; cyano; —C(O)R^{g}, —C(O)OR^{g}; —OC(O)R^{g}; —C(O)SR^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; or —NR^{h}S(O)ₙR^{j}; or

(ii) C₁-C₁₂ (e.g., C₁-C₆) alkyl or C₁-C₁₂ (e.g., C₁-C₆) haloalkyl, each of which is optionally substituted with from 1-5 R^{d}; or

(iii) C₂-C₁₂ (e.g., C₂-C₆) alkenyl or C₂-C₁₂ (e.g., C₂-C₆) alkynyl, each of which is optionally substituted with from 1-5 R^{e}; or

(iv) C₇-C₂₀ aralkyl, optionally substituted with from 1-10 R^{f}; and R^{a2}, R^{a4}, R^{a5}, and R^{a6} can each be hydrogen.

In certain embodiments, R^{a3} can be nitro, and R^{a2}, R^{a4}, R^{a5}, and R^{a6} can each be hydrogen.

In certain embodiments, R^{a3} can be NR^{b}R^{c}, and R^{a2}, R^{a4}, R^{a5}, and R⁶ can each be hydrogen. In these embodiments, R^{b} and R^{c} can be as defined anywhere herein. In certain embodiments, one of R^{b} and R^{c} can be hydrogen, and the other can be a substituent other than hydrogen (e.g., a cyclic substituent). In other embodiments, both R^{b} and R^{c} can each be a substituent other than hydrogen. In these embodiments, R^{b} and R^{c} can be the same or different.

In certain embodiments, one of R^{b} and R^{c} can be hydrogen, and the other can be C₃-C₁₀ cycloalkyl or heterocyclyl including 3-10 atoms, each of which is optionally substituted with from 1-10 R^{f}.

For example, one of R^{b} and R^{c} can be hydrogen, and the other can be cyclohexyl or cyclohexyl substituted with from 1-5 R^{f} (e.g., C₁-C₄ alkyl, e.g., CH₃). An exemplary substituted cyclohexyl group is 3,3,5,5-tetramethylcyclohex-1-yl.

As another example, one of R^{b} and R^{c} can be hydrogen, and the other can be piperidyl (e.g., 4-piperidyl), optionally substituted with from 1-2 R^{f} (e.g., C₇-C₂₀ aralkyl, e.g., benzyl). An exemplary piperidyl moiety is 1-(benzylsulfonyl)-4-piperidyl.

In certain embodiments, one of R^{b} and R^{c} can be C₃-C₂₀ cycloalkyl, optionally substituted with from 1-10 R^{f}, and the other can be —S(O)ₙR^{j}, in which R^{j} is NR^{b}R^{c} (e.g., NH₂).

One of R^{b} and R^{c} can be cyclohexyl or cyclohexyl optionally substituted with from 1-5 R^{f}, and the other can be —S(O)₂NH₂.

In all embodiments, one of R¹, R², R³, or R⁴ has formula (II):

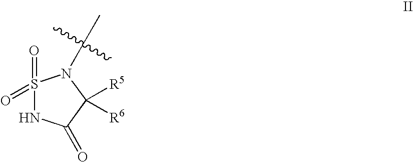

In some embodiments, R⁵ and R⁶ can each be, independently, hydrogen, fluoro, or C₁-C₄ alkyl. For example, R⁵ and R⁶ can both be hydrogen. As another example, one of R⁵ and R⁶ can be hydrogen, and the other can be C₁-C₄ alkyl (e.g., CH₃).

In some embodiments, the PTP1b inhibitor compounds can have formula (IV), in which R³ has formula (II):

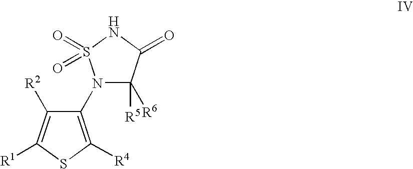

and R¹, R², and R⁴; and R⁵ and R⁶ can be as defined elsewhere.

In all embodiments, two of R¹, R², R³, or R⁴ (e.g., R² and R⁴) are each, independently:

(i) hydrogen; or

(ii) halo; NR^{b}R^{c}; nitro; azido; hydroxy; C₁-C₁₂ alkoxy or C₁-C₁₂ thioalkoxy, each of which is optionally substituted with 1-5 R^{d}; C₁-C₁₂ haloalkoxy; C₆-C₁₆ aryloxy, C₆-C₁₆ thioaryloxy, heteroaryloxy including 5-20 atoms, or thioheteroaryloxy including 5-20 atoms, each of which is optionally substituted with 1-5 R^{a}; C₂-C₁₂ alkenyloxy or C₂-C₁₂ alkynyloxy, each of which is optionally substituted with 1-5 R^{e}; C₃-C₁₆ cycloalkyloxy, C₃-C₁₆ cycloalkenyloxy, heterocyclyloxy including 3-16 atoms, heterocycloalkenyloxy including 3-16 atoms, C₇-C₂₀ aralkoxy, or heteroaralkoxy including 6-20 atoms, each of which is optionally substituted with 1-5 R^{f}; mercapto; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or

(iii) C₁-C₁₂ alkyl or C₁-C₁₂ haloalkyl; each of which is optionally substituted with from 1-5 R^{d}; or

(iv) C₂-C₂₀ alkenyl or C₂-C₂₀ alkynyl, each of which is optionally substituted with from 1-10 R^{e}.

In some embodiments, one of these two substituents (i.e., one of R¹, R², R³, or R⁴, e.g., R² or R⁴, preferably R⁴) can be hydrogen, and one of these two substituents (i.e., one of R¹, R², R³, or R⁴, e.g., R² or R⁴, preferably R⁴) can be:

(ii) halo; NR^{b}R^{c}; nitro; azido; hydroxy; C₁-C₁₂ alkoxy or C₁-C₁₂ thioalkoxy, each of which is optionally substituted with 1-5 R^{d}; C₁-C₁₂ haloalkoxy; C₆-C₁₆ aryloxy, C₆-C₁₆ thioaryloxy, heteroaryloxy including 5-20 atoms, or thioheteroaryloxy including 5-20 atoms, each of which is optionally substituted with 1-5 R^{a}; C₂-C₁₂ alkenyloxy or C₂-C₁₂ alkynyloxy, each of which is optionally substituted with 1-5 R^{e}; C₃-C₁₆ cycloalkyloxy, C₃-C₁₆ cycloalkenyloxy, heterocyclyloxy including 3-16 atoms, heterocycloalkenyloxy including 3-16 atoms, C₇-C₂₀ aralkoxy, or heteroaralkoxy including 6-20 atoms, each of which is optionally substituted with 1-5 R^{f}; mercapto; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or

(iii) C₁-C₁₂ alkyl or C₁-C₁₂ haloalkyl; each of which is optionally substituted with from 1-5 R^{d}; or

(iv) C₂-C₂₀ alkenyl or C₂-C₂₀ alkynyl, each of which is optionally substituted with from 1-10 R^{e}.

In some embodiments, two of R¹, R², R³, or R⁴ (e.g., R² and R⁴) can be hydrogen.

In some embodiments, two of R¹, R², R^{3,} or R⁴ (e.g., R² and R⁴) can each be, independently:

(ii) halo; NR^{b}R^{c}; nitro; azido; hydroxy; C₁-C₁₂ alkoxy or C₁-C₁₂ thioalkoxy, each of which is optionally substituted with 1-5 R^{d}; C₁-C₁₂ haloalkoxy; C₆-C₁₆ aryloxy, C₆-C₁₆ thioaryloxy, heteroaryloxy including 5-20 atoms, or thioheteroaryloxy including 5-20 atoms, each of which is optionally substituted with 1-5 R^{a}; C₂-C₁₂ alkenyloxy or C₂-C₁₂ alkynyloxy, each of which is optionally substituted with 1-5 R^{e}; C₃-C₁₆ cycloalkyloxy, C₃-C₁₆ cycloalkenyloxy, heterocyclyloxy including 3-16 atoms, heterocycloalkenyloxy including 3-16 atoms, C₇-C₂₀ aralkoxy, or heteroaralkoxy including 6-20 atoms, each of which is optionally substituted with 1-5 R^{f}; mercapto; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or

(iii) C₁-C₁₂ alkyl or C₁-C₁₂ haloalkyl; each of which is optionally substituted with from 1-5 R^{d}; or

(iv) C₂-C₂₀ alkenyl or C₂-C₂₀ alkynyl, each of which is optionally substituted with from 1-10 R^{e}.

In some embodiments, two of R¹, R², R³, or R⁴ (e.g., R² and R⁴) can each be, independently, hydrogen; halo; cyano, —C(O)OR^{g}; —C(O)NR^{b}R^{c}; or C₁-C₁₂ (e.g., C₁-C₆ or C₁-C₄) alkyl or C₁-C₁₂ (e.g., C₁-C₆ or C₁-C₄) haloalkyl, each of which is optionally substituted with from 1-5 (e.g., 1-3, 1-2, or 1) R^{d}.

In certain embodiments, R² can be hydrogen; halo; C₁-C₁₂ (e.g., C₁-C₆ or C₁-C₄) alkyl (e.g., CH₃); or C₁-C₁₂ (e.g., C₁-C₆ or C₁-C₄) haloalkyl (e.g., CF₃).

In certain embodiments, R⁴ can be hydrogen; halo; cyano; —C(O)OR^{g}; —C(O)NR^{b}R^{c}; C₁-C₁₂ (e.g., C₁-C₆, C₁-C₄) haloalkyl (e.g., CF₃); or C₁-C₁₂ (e.g., C₁-C₆, C₁-C₄) alkyl, optionally substituted with from 1-3 R^{d} (e.g., OH, e.g., R⁴ can be —CH₂OH).

In certain embodiments, one of R² and R⁴ (e.g., R⁴) can be hydrogen, and the other (e.g., R²) can be halo; cyano, —C(O)OR^{g}; —C(O)NR^{b}R^{c}; or C₁-C₁₂ alkyl or C₁-C₁₂ haloalkyl, each of which is optionally substituted with from 1-5 R^{d}.

For example, R² can be C₁-C₄ alkyl (e.g., CH₃), and R⁴ can be hydrogen; or R² can be halo (e.g., chloro or bromo), and R⁴ can be hydrogen.

In certain embodiments, R² and R⁴ can both be hydrogen.

In certain embodiments, R² and R⁴ can both be a substituent other than hydrogen. For example, R² and R⁴ can both be halo (e.g., chloro).

In some embodiments, the PTP1b inhibitor compounds can have formula (IV), in which the following definitions apply:

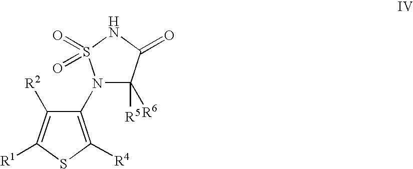

R¹is C₆-C₁₆ aryl or heteroaryl including 5-16 atoms, each of which is optionally substituted with from 1-10 R^{a};

R² and R⁴ are each, independently:

(i) hydrogen; or

(ii) halo; NR^{b}R^{c}; nitro; azido; hydroxy; C₁-C₁₂ alkoxy or C₁-C₁₂ thioalkoxy, each of which is optionally substituted with 1-5 R^{d}; C₁-C₁₂ haloalkoxy; C₆-C₁₆ aryloxy, C₆-C₁₆ thioaryloxy, heteroaryloxy including 5-20 atoms, or thioheteroaryloxy including 5-20 atoms, each of which is optionally substituted with 1-5 R^{a}; C₂-C₁₂ alkenyloxy or C₂-C₁₂ alkynyloxy, each of which is optionally substituted with 1-5 R^{e}; C₃-C₁₆ cycloalkyloxy, C₃-C₁₆ cycloalkenyloxy, heterocyclyloxy including 3-16 atoms, heterocycloalkenyloxy including 3-16 atoms, C₇-C₂₀ aralkoxy, or heteroaralkoxy including 6-20 atoms, each of which is optionally substituted with 1-5 R^{f}; mercapto; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or

(ii) C₁-C₁₂ alkyl or C₁-C₁₂ haloalkyl; each of which is optionally substituted with from 1-5 R^{d}; or

(iii) C₂-C₂₀ alkenyl or C₂-C₂₀ alkynyl, each of which is optionally substituted with from 1-10 R^{e}; and

R^{a}, R^{a′}, R^{b}, R^{c}, R^{d}, R^{e}, R^{f}, R^{g}, R^{h}, RⁱR^{j}, R⁵, and R⁶ can be as defined anywhere herein.

Embodiments can include any one or more of the features described herein.

In certain embodiments, the PTP1b inhibitor compounds can have formula (V), in which the following definitions apply:

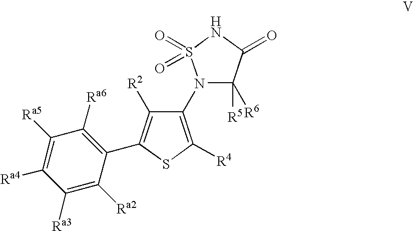

R² and R⁴ are each, independently:

(i) hydrogen; or

(ii) halo; NR^{b}R^{c}; nitro; azido; hydroxy; C₁-C₁₂ alkoxy or C₁-C₁₂ thioalkoxy, each of which is optionally substituted with 1-5 R^{d}; C₁-C₁₂ haloalkoxy; C₆-C₁₆ aryloxy, C₆-C₁₆ thioaryloxy, heteroaryloxy including 5-20 atoms, or thioheteroaryloxy including 5-20 atoms, each of which is optionally substituted with 1-5 R^{a}; C₂-C₁₂ alkenyloxy or C₂-C₁₂ alkynyloxy, each of which is optionally substituted with 1-5 R^{e}; C₃-C₁₆ cycloalkyloxy, C₃-C₁₆ cycloalkenyloxy, heterocyclyloxy including 3-16 atoms, heterocycloalkenyloxy including 3-16 atoms, C₇-C₂₀ aralkoxy, or heteroaralkoxy including 6-20 atoms, each of which is optionally substituted with 1-5 R^{f}; mercapto; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or

(ii) C₁-C₁₂ alkyl or C₁-C₁₂ haloalkyl; each of which is optionally substituted with from 1-5 R^{d}; or

(iii) C₂-C₂₀ alkenyl or C₂-C₂₀ alkynyl, each of which is optionally substituted with from 1-10 R^{e};

R^{a2}, R^{a3}, R^{a4}, R^{a5}, and R^{a6} are each, independently:

(i) halo; NR^{b}R^{c}(e.g., NH₂, monosubstituted or disubstituted amino in which one or both, respectively, of R^{b} and R^{c} is other than hydrogen); nitro; hydroxy; C₁-C₁₂ (e.g., C₁-C₆) alkoxy or C₁-C₁₂ (e.g., C₁-C₆) haloalkoxy (e.g., OCF₃), each of which is optionally substituted with from 1-10 R^{d}; C₆-C₁₀ aryloxy or heteroaryloxy including 5-10 atoms, each of which is optionally substituted with from 1-5 R^{a} or R^{a′}; C₂-C₁₂ (e.g., C₂-C₆) alkenyloxy or C₂-C₁₂ (e.g., C₂-C₆) alkynyloxy, each of which is optionally substituted with 1-5 R^{e}; C₇-C₁₂ aralkoxy, heteroaralkoxy including 6-12 atoms, C₃-C₈ cycloalkoxy, C₃-C₈ cycloalkenyloxy, heterocyclyloxy including 3-8 atoms, or heterocycloalkenyloxy including 3-8 atoms, each of which is optionally substituted with from 1-10 R^{f}; mercapto; C₁-C₁₂ (e.g., C₁-C₆) thioalkoxy or C₁-C₁₂ (e.g., C₁-C₆) thiohaloalkoxy, each of which is optionally substituted with from 1-5 R^{d}; C₆-C₁₀ thioaryloxy or thioheteroaryloxy including 5-10 atoms, each of which is optionally substituted with from 1-5 R^{a} or R^{a′}; C₂-C₁₂ (e.g., C₂-C₆) thioalkenyloxy or C₂-C₁₂ (e.g., C₂-C₆) thioalkynyloxy, each of which is optionally substituted with 1-5 R^{e}; C₇-C₁₂ thioaralkoxy, thioheteroaralkoxy including 6-12 atoms, C₃-C₈ thiocycloalkoxy, C₃-C₈ thiocycloalkenyloxy, thioheterocyclyloxy including 3-8 atoms, or thioheterocycloalkenyloxy including 3-8 atoms, each of which is optionally substituted with from 1-10 R^{f}; cyano; —C(O)R^{g}, —C(O)OR^{g}; —OC(O)R^{g}; —C(O)SR^{g}; —C(O)R^{b}R^{c}; NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; or —NR^{h}S(O)ₙRⁱ; or

(ii) C₁-C₁₂ (e.g., C₁-C₆) alkyl or C₁-C₁₂ (e.g., C₁-C₆) haloalkyl, each of which is optionally substituted with from 1-5 R^{d}; or

(iii) C₂-C₁₂ (e.g., C₂-C₆) alkenyl or C₂-C₁₂ (e.g., C₂-C₆) alkynyl, each of which is optionally substituted with from 1-5 R^{e}; or

(iv) C₇-C₂₀ aralkyl, optionally substituted with from 1-10 R^{f}; or

(v) hydrogen; and

R^{a}, R^{a′}, R^{c}, R^{d}, R^{e}, R^{f}, R^{g}, RⁱR^{j}, R⁵, and R⁶ can be as defined anywhere herein.

Embodiments can include any one or more of the features described herein.

It is understood that the actual electronic structure of some chemical entities cannot be adequately represented by only one canonical form (i.e. Lewis structure). While not wishing to be bound by theory, the actual structure can instead be some hybrid or weighted average of two or more canonical forms, known collectively as resonance forms or structures. Resonance structures are not discrete chemical entities and exist only on paper. They differ from one another only in the placement or “localization” of the bonding and nonbonding electrons for a particular chemical entity. It can be possible for one resonance structure to contribute to a greater extent to the hybrid than the others. Thus, the written and graphical descriptions of the embodiments of the present invention are made in terms of what the art recognizes as the predominant resonance form for a particular species.

The compounds described herein can be synthesized according to methods described herein and/or conventional, organic chemical synthesis methods from commercially available starting materials and reagents. The compounds described herein can be separated from a reaction mixture and further purified by a method such as column chromatography, high-pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

In certain embodiments, the PTP1b inhibitor compounds described herein can generally be prepared as delineated in Scheme A below.

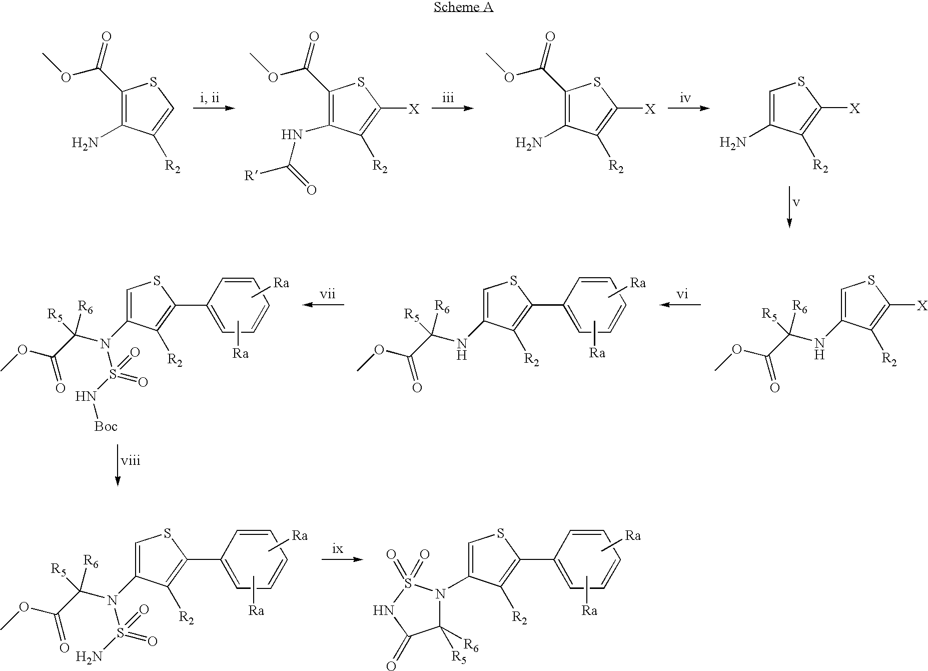

i) (CF₃CO)₂O or (CH₃CO)₂O, DIPEA, DCM; ii) bromination or chlorination; iii) NaOH/EtOH or HCl/MeOH; iv)a) NaOH, EtOH b) Conc. HCl; v) K₂CO₃, methyl bromoacetate, DMF; vi) crossing coupling reactions; vii) sulfurisocyanatidic chloride, t-BuOH, DIPEA, DCM; viii) TFA, DCM; ix) NaH, THF.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e.g., carbon-carbon bonds, carbon-nitrogen bonds such as amide bonds) wherein bond rotation is restricted about that particular linkage, e.g. restriction resulting from the presence of a ring or double bond. Accordingly, all cis/trans and E/Z isomers and rotational isomers are expressly included in the present invention. The compounds of this invention may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

The compounds of this invention include the compounds themselves, as well as their salts and their prodrugs, if applicable. A salt, for example, can be formed between an anion and a positively charged substituent on a compound described herein. In some embodiments, the positively charged substituent can be a protonated or quaternized amino group having the general formula:

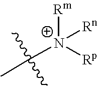

in which each of R^{m}, Rⁿ, and R^{p} can be, independently of one another:

(i) hydrogen; or

(ii) C₁-C₂₀ alkyl or C₁-C₂₀ haloalkyl, each of which is optionally substituted with from 1-10 R^{d}; or

(iii) C₂-C₂₀ alkenyl or C₂-C₂₀ alkynyl, each of which is optionally substituted with from 1-10 R^{e}; or

(iv) C₃-C₂₀ cycloalkyl, C₃-C₂₀ cycloalkenyl, heterocyclyl including 3-20 atoms, or heterocycloalkenyl including 3-20 atoms, C₇-C₂₀ aralkyl, or heteroaralkyl including 6-20 atoms, each of which is optionally substituted with from 1-10 R^{f}; or

(v) C₆-C₁₈ aryl or heteroaryl including 5-16 atoms, each of which is optionally substituted with from 1-10 R^{a}.

### Suitable anions include chloride, bromide, iodide, sulfate, nitrate, phosphate, citrate, methanesulfonate, trifluoroacetate, and acetate.

Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active compounds.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization. Salt forms of the compounds of any of the formulae herein can be amino acid salts of carboxy groups (e.g. L-arginine, -lysine, -histidine salts).

The term “pharmaceutically acceptable carrier or adjuvant” refers to a carrier or adjuvant that may be administered to a subject (e.g., a patient), together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

In general, the compounds described herein can be used for treating (e.g., controlling, ameliorating, preventing, delaying the onset of, or reducing the risk of developing) one or more diseases, disorders, conditions or symptoms mediated by PTP1B (e.g., type 2 diabetes, obesity, metabolic syndromes). A disorder or physiological condition that is mediated by PTP1b refers to a disorder or condition wherein PTP1b can trigger the onset of the condition, or where inhibition of a particular PTPase can affect signaling in such a way so as to treat, control, ameliorate, prevent, delay the onset of, or reduce the risk of developing the disorder or condition. Examples of such disorders include, but are not limited to, type I and type 2 diabetes, obesity, cancer, autoimmune diseases, allergic disorders, acute and chronic inflammation, metabolic syndrome, and osteoporosis.

The compounds described herein generally have an inhibition constant Ki of less than about 500 μM (e.g., less than about 400 μM, less than about 300 μM, less than about 200 μM, less than about 100 μM, less than 50 μM, less than 1 μM). In certain embodiments, compounds described herein can have an inhibition constant Ki of from about 500 μM to about 1 μM. In certain embodiments, compounds described herein can have an inhibition constant Ki of about 100 nM.

In some embodiments, the compounds described herein can be coadministered with one or more other threapeutic agents. In certain embodiments, the additional agents may be administered separately, as part of a multiple dose regimen, from the compounds of this invention (e.g., sequentially, e.g., on different overlapping schedules with the administration of one or more compounds of formula (I)). Alternatively, these agents may be part of a single dosage form, mixed together with the compounds of this invention in a single composition. In still another embodiment, these agents can be given as a separate dose that is administered at about the same time that one or more compounds of formula (I) are administered (e.g., simultaneously with the administration of one or more compounds of formula (I)). When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen.

Other therapeutic agents can include, e.g., insulin and insulin analogues and mimetics; PPAR agonists (e.g., pioglitazone, rosiglitazone), statins (e.g., simvastatin, e.g., Zocor; atorvastatin calcium (e.g., lipitor); ACE inhibitors (e.g., lisinopril), and ARB inhibitors.

The compounds and compositions described herein can, for example, be administered orally, parenterally (e.g., subcutaneously, intracutaneously, intravenously, intramuscularly, intraarticularly, intraarterially, intrasynovially, intrasternally, intrathecally, intralesionally and by intracranial injection or infusion techniques), by inhalation spray, topically, rectally, nasally, buccally, vaginally, via an implanted reservoir, by injection, subdermally, intraperitoneally, transmucosally, or in an ophthalmic preparation, with a dosage ranging from about 0.01 mg/Kg to about 1000 mg/Kg, (e.g., from about 0.01 to about 100 mg/kg, from about 0.1 to about 100 mg/Kg, from about I to about 100 mg/Kg, from about 1 to about 10 mg/kg) every 4 to 120 hours, or according to the requirements of the particular drug. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardsley, N.Y., 537 (1970). In certain embodiments, the compositions are administered by oral administration or administration by injection. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

The compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the compositions of this invention is useful when the desired treatment involves areas or organs readily accessible by topical application. For application topically to the skin, the composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The compositions of this invention may also be topically applied to the lower intestinal tract by rectal suppository formulation or in a suitable enema formulation.

Topically-transdermal patches are also included in this invention. Also within the invention is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device.

The compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compound of the formulae herein and an additional agent (e.g., a therapeutic agent) can be administered using any of the routes of administration described herein. In some embodiments, a composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal),can also be used for delivery of the compounds and compositions delineated herein.

The invention will be further described in the following examples. It should be understood that these examples are for illustrative purposes only and are not to be construed as limiting this invention in any manner.

### EXAMPLES

### Example 1

### 5-(5-phenyl-3-thienyl)-1,2,5-thiadiazolidin-3-one 1,1-dioxide

Step 1A: Methyl 3-amino-5-phenylthiophene-2-carboxylate (468 mg, 2 mmole) was suspended in 16 mL EtOH in a microwave vessel. 2.7 mL of 15% aqueous NaOH (10 mmole) was added and the reaction heated to 100° C. for 30 minutes. Concentrated HCl was added until the reaction mixture was acidic and stirred at room temperature for 30 minutes (CO₂ evolution observed). The reaction mixture was partitioned between EtOAc and concentrated aqueous sodium bicarbonate. The aqueous phase was extracted with EtOAc and the combined organics washed with water, brine and dried (MgSO₄). Filtration and evaporation gave 332 mg of 3-amino-5-phenylthiophene (95%) as a pale yellow solid.

¹H NMR (400 MHz, CHLOROFORM-D) δ ppm 6.14 (d, J=1.65 Hz, 3 H) 6.89 (d, J=1.65 Hz, 1 H) 7.23-7.29 (m, 1 H) 7.32-7.38 (m, 2 H) 7.53-7.57 (m, 2 H).

Step 1B: 3-amino-5-phenylthiophene (330 mg, 1.9 mmole) was dissolved in 10 mL DMF and potassium carbonate (650 mg, 4.7 mmole) was added followed by methyl bromoacetate (182 μL, 2.0 mmole). The reaction mixture was heated to 60° C. for 2 hours, after which LC/MS analysis showed complete reaction. The mixture was cooled to room temperature, then water was added and extracted with EtOAc (3×30 mL). The combined organic phases were washed with water, brine, and dried (MgSO₄). Filtration and evaporation gave 387 mg of methyl 2-(5-phenylthiophen-3-ylamino)acetate (82%) as a brown oil.

1H NMR (400 MHz, CHLOROFORM-D) δ ppm 3.80 (s, 3 H) 3.90 (s, 2 H) 5.91 (d, J=1.77 Hz, 1 H) 6.90 (d, J=1.77 Hz, 1 H) 7.24-7.30 (m, 1 H) 7.33-7.39 (m, 2 H) 7.53-7.57 (m, 2 H)

Step 1C: Chlorosulfonyl isocyanate (164 μL, 1.88 mmole) was dissolved in 3 mL dichloromethane and tBuOH (164 μL, 2.8 mmole) was added and stirred at room temperature for 30 minutes. This solution was added dropwise to an 8 mL dichloromethane solution of methyl 2-(5-phenylthiophen-3-ylamino)acetate (387 mg, 1.6 mmole) and diisopropylethyl amine (0.42 mL, 2.3 mmole). The mixture was stirred at room temperature for 1 hour after which time the reaction was deemed complete by LC/MS. The reaction diluted with dichloromethane and washed with dilute aqueous HCl, water and dried (MgSO₄). Filtration and evaporation gave the crude product which was purified by flash chromatography using a gradient of 5% to 40% EtOAc in hexane. 366 mg of methyl 2-((N-(tert-butoxycarbonyl)sulfamoyl)(5-phenylthiophen-3-yl)amino)acetate (54%) was obtained as a yellow oil that crystallized on standing.

1H NMR (400 MHz, CHLOROFORM-D) δ ppm 1.49 (s, 9 H) 3.78 (s, 3 H) 4.62 (s, 2 H) 7.29-7.35 (m, 3 H) 7.36-7.41 (m, 2 H) 7.53-7.58 (m, 2 H)

Step 1D: Methyl 2-((N-(tert-butoxycarbonyl)sulfamoyl)(5-phenylthiophen-3-yl)amino)acetate (365 mg, 0.86 mmole) was dissolved in 5 mL dichloromethane and 1.5 mL trifluoroacetic acid was added and the mixture was stirred at room temperature for 2 hours. Solvents were evaporated and then co-evaporated three times with dichloromethane. Methyl 2-((5-phenylthiophen-3-yl)(sulfamoyl)amino)acetate was obtained in quantitative yield as a tacky brown oil.

1H NMR (400 MHz, CHLOROFORM-D) δ ppm 3.85 (s, 3 H) 4.48 (s, 2 H) 5.18 (s, 2 H) 7.15 (d, J=1.52 Hz, 1 H) 7.29-7.34 (m, 1 H) 7.36 (d, J=1.52 Hz, 1 H) 7.36-7.42 (m, 2 H) 7.55-7.59 (m, 2 H)

Methyl 2-((5-phenylthiophen-3-yl)(sulfamoyl)amino)acetate (129 mg, 0.4 mmole) was dissolved in 2 mL THF and NaH (47 mg of 60% dispersion in mineral oil, 1.2 mmole) added (vigorous bubbling). The reaction mixture became a suspension almost immediately after addition of NaH. After 1 hour, the reaction mixture was carefully quenched with water (15 mL) and a few mLs of 1N NaOH and extracted-with 1:1 hexane:EtOAc. The aqueous phase was made acidic to pH=1 with 2N HCl and then extracted with EtOAc (2×15 mL). The combined organic layers were washed with brine and dried to give a pale yellow solid. NMR analysis revealed that mineral oil was still present in the crude product. The solid was triturated with hexane then filtered, washed with hexane and suction dried to give 60 mg of the title compound as a pale green solid (50%).

1H NMR (400 MHz, MeOD) δ ppm 4.64 (s, 2 H) 7.05 (d, J=1.65 Hz, 1 H) 7.36-7.42 (m, 1 H) 7.44-7.50 (m, 2 H) 7.51 (d, J=1.65 Hz, 1 H) 7.67-7.73 (m, 2 H)

HRMS: calcd for C₁₂H10N2O3S2+H+, 295.02056; found (ESI-FTMS, [M+H]1+), 295.0204.

### Example 2

### 5-(4-chloro-5-phenyl-3-thienyl)-1,2,5-thiadiazolidin-3-one 1,1-dioxide

Step 2A: Methyl 3-amino-2-thiophenecarboxylate (15.72 g, 100 mmole) was dissolved in 100 mL pyridine and cooled in an ice bath. Acetyl chloride (7.5 mL, 105 mmole) was added dropwise and the cold bath was removed and the resulting suspension was stirred at room temperature for 1 hour. The solvent was evaporated to a red semisolid and then triturated with 200 mL water. The suspension was filtered, washed with water and suction dried to give 18.23 g (91%) of methyl 3-acetamidothiophene-2-carboxylate as a tan-colored solid.

1H NMR (400 MHz, DMSO-D6) δ ppm 2.17 (s, 3 H) 3.84 (s, 3 H) 7.86-7.94 (m, 2 H) 9.99 (s, 1 H)

Step 2B: Methyl 3-acetamidothiophene-2-carboxylate (18.23 g, 91.5 mmole) was dissolved in 100 mL chloroform and sulfuryl chloride (16.2 mL, 201.3 mmole) was added dropwise and the solution heated to reflux for 2 hours. The solvent was evaporated and the dark red semisolid residue was triturated with 150 mL ether. The suspension was filtered, washed with ether and dried to give 15.4 g (63%) methyl 3-acetamido-4,5-dichlorothiophene-2-carboxylate as a light purple colored solid.

1H NMR (400 MHz, CHLOROFORM-D) δ ppm 2.23 (s, 3 H) 3.87 (s, 3 H) 8.20 (s, 1 H)

Step 2C: Methyl 3-acetamido-4,5-dichlorothiophene-2-carboxylate (536 mg, 2.0 mmole), phenylboronic acid (292 mg, 2.4 mmole), KF (349 mg, 6.0 mmole), and Pd(PPh₃)₄ (116 mg, 5 mol %) were charged in a microwave vessel and 10 mL THF added. The vessel was capped and heated to 150° C. for 30 minutes. The reaction mixture was diluted with ethyl acetate, and washed with saturated sodium bicarbonate, brine, and dried (MgSO₄). Filtration and evaporation gave the crude product which was purified by flash chromatography using a gradient of 15% to 65% EtOAc in hexane. Obtain 407 mg (65%) methyl 3-acetamido-4-chloro-5-phenylthiophene-2-carboxylate as a pale yellow solid.

1H NMR (400 MHz, DMSO-D6) δ ppm 2.07 (s, 3 H) 3.81 (s, 3 H) 7.50-7.59 (m, 3 H) 7.71 (dd, J=7.96, 1.64 Hz, 2 H)

Step 2D: Methyl 3-acetamido-4-chloro-5-phenylthiophene-2-carboxylate was deacylated and decarboxylated as in Example 1, Step 1A, to give 4-chloro-5-phenylthiophen-3-amine in 91% yield as a yellow oil.

1H NMR (400 MHz, CHLOROFORM-D) δ ppm 3.81 (s, 2 H) 6.23 (s, 1 H) 7.32-7.38 (m, 1 H) 7.39-7.45 (m, 2 H) 7.63-7.67 (m, 2 H)

Step 2E: 4-chloro-5-phenylthiophen-3-amine was alkylated as in Example 1, Step 1B, to give methyl 2-(4-chloro-5-phenylthiophen-3-ylamino)acetate in 65% yield as a yellow oil.

1H NMR (400 MHz, CHLOROFORM-D) δ ppm 3.81 (s, 3 H) 3.94 (d, J=5.81 Hz, 2 H) 4.55 (t, J=5.81 Hz, 1 H) 5.91 (s, 1 H) 7.32-7.38 (m, 1 H) 7.39-7.46 (m, 2 H) 7.63-7.67 (m, 2 H)

Step 2F: Methyl 2-(4-chloro-5-phenylthiophen-3-ylamino)acetate was sulfonylated as in Example 1, Step 1C, to give methyl 2-((N-(tert-butoxycarbonyl)sulfamoyl)(4-chloro-5-phenylthiophen-3-yl)amino)acetate in 87% yield as a colorless oil.

1H NMR (400 MHz, CHLOROFORM-D) δ ppm 1.50 (s, 9 H) 3.77 (s, 3 H) 4.61 (s, 2 H) 7.32 (s, 1 H) 7.39-7.49 (m, 3 H) 7.60-7.65 (m, 2 H) 7.80 (s, 1 H)

Step 2G: Methyl 2-((N-(tert-butoxycarbonyl)sulfamoyl)(4-chloro-5-phenylthiophen-3-yl)amino)acetate was deprotected as in Example 1, Step 1D, to give methyl 2-((4-chloro-5-phenylthiophen-3-yl)(sulfamoyl)amino)acetate in quantitative yield as a pale yellow oil that solidifies on standing.

1H NMR (400 MHz, CHLOROFORM-D) δ ppm 3.79 (s, 3 H) 4.42 (s, 2 H) 5.23 (s, 2 H) 7.36-7.48 (m, 3 H) 7.62-7.66 (m, 2 H) 7.72 (s, 1 H)

Step 2H: Methyl 2-((4-chloro-5-phenylthiophen-3-yl)(sulfamoyl)amino)acetate was cyclized as in Example 1, Step 1E, to give the final compound in 30% yield as an off-white solid.

1H NMR (400 MHz, DMSO-D6) δ ppm 4.36 (s, 2 H) 7.41-7.48 (m, 1 H) 7.52 (t, J=7.45 Hz, 2 H) 7.62-7.66 (m, 2 H) 7.87 (s, 1 H)

HRMS: calcd for C₁₂H9ClN2O3S2+H+, 328.98159; found (ESI-FTMS, [M+H]1+), 328.982.

### Example 3

### 5-(2,4-dichloro-5-phenyl-3-thienyl)-1,2,5-thiadiazolidin-3-one 1,1-dioxide

Step 3A: Methyl 2-((5-phenylthiophen-3-yl)(sulfamoyl)amino)acetate (51 mg, 0.14 mmole) was dissolved in 2 mL chloroform and sulfuryl chloride (14 μL, 0.17 mmole) was added and the solution heated to 60° C. for 30 minutes. The reaction mixture was cooled to room temperature, diluted with CH₂Cl₂, washed with saturated sodium bicarbonate solution, and dried (MgSO₄). Filtration and evaporation gave the crude product which was purified by flash chromatography using a gradient of 10% to 40% EtOAc in hexane. 42 mg (78%) of methyl 2-((2,4-dichloro-5-phenylthiophen-3-yl)(sulfamoyl)amino)acetate was obtained as a colorless oil.

1H NMR (400 MHz, CHLOROFORM-D) δ ppm 3.80 (s, 3 H) 4.30 (d, J=18.19 Hz, 1 H) 4.55 (d, J=18.19 Hz, 1 H) 5.34 (s, 2 H) 7.38-7.49 (m, 3 H) 7.55-7.63 (m, 2 H)

Step 3B: Methyl 2-((5-phenylthiophen-3-yl)(sulfamoyl)amino)acetate was cyclized as in Example 1, Step 1E. The crude product was purified by preparative thin layer chromatography to give the final compound in 63% yield as a white solid.

1H NMR (400 MHz, DMSO-D6) δ ppm 3.98-4.18 (m, 2 H) 7.42-7.56 (m, 3 H) 7.62 (d, J=7.33 Hz, 2 H)

ESI-MS: m/e═361 [M−H]⁻.

### Example 4

### 5-(4-methyl-5-phenyl-3-thienyl)-1,2,5-thiadiazolidin-3-one 1,1-dioxide

Step 4A: Methyl 3-amino-4-methylthiophene-2-carboxylate (1 g, 6.36 mmol) and diisopropylethylamine (3.3 mL, 19 mmol) were dissolved in 30 mL of DCM. Trifluoroacetic anhydride (1.35 mL, 9.54 mmol) was then added dropwise. The mixture was stirred at room temperature for 2 hours. Solvent was evaporated and 200 mL of water was then added. A precipitate was formed, filtered and washed with water. Methyl 4-methyl-3-(2,2,2-trifluoroacetamido)thiophene-2-carboxylate (1.7 g, >95% yield) was obtained as a beige solid.

1H NMR (400 MHz, CHLOROFORM-D) δ ppm 2.21 (s, 3 H) 3.89 (s, 3 H) 7.27 (s, 1 H) 9.65 (s, 1 H); ESI-MS: m/e=266.17 [M−H]⁻.

Step 4B: Methyl 4-methyl-3-(2,2,2-trifluoroacetamido)thiophene-2-carboxylate (3.9 g, 14.6 mmol) was dissolved in 50 mL acetic acid. N-bromosuccinamide (5.2 g, 29.2 mmol) was then added. The mixtured was stirred at 80° C. overnight. Water (200 mL) and brine (100 mL) were added. The precipitate was filtered and washed with water. Methyl 5-bromo-4-methyl-3-(2,2,2-trifluoroacetamido)thiophene-2-carboxylate (1.97 g, 39% yield) was obtained as a beige solid.

1H NMR (400 MHz, CHLOROFORM-D) δ ppm 2.15 (s, 3 H) 3.89 (s, 3 H) 9.62 (s, 1 H); ESI-MS: m/e=344.11 [M−H]⁻.

Step 4C: Methyl 5-bromo-4-methyl-3-(2,2,2-trifluoroacetamido)thiophene-2-carboxylate (200 mg, 0.58 mmol), Pd(PPh₃)₄ (67 mg, 0.058 mmol), phenylboronic acid (106 mg, 0.87 mmol), and KF (101 mg, 1.74 mmol) were mixed in 4 mL of THF. The mixture was heated at 140° C. in microwave (Personal Chemistry) for 30 minutes. The mixture was filtered through a plug of celite and solvent was evaporated. The crude product was purified by flash column chromatography. Methyl 4-methyl-5-phenyl-3-(2,2,2-trifluoroacetamido)thiophene-2-carboxylate (103 mg, 52% yield) was obtained as an orange oil.

1H NMR (400 MHz, CHLOROFORM-D) δ ppm 2.19 (s, 3 H) 3.90 (s, 3 H) 7.39-7.50 (m, 5 H) 9.67 (s, 1 H); ESI-MS: m/e=342.20 [M−H]⁻.

Step 4D: Methyl 4-methyl-5-phenyl-3-(2,2,2-trifluoroacetamido)thiophene-2-carboxylate (103 mg, 0.3 mmol) was dissolved in 3 mL of EtOH. NaOH (15% solution, 0.4 mL) was then added. The mixture was heated at 100° C. in microwave for 30 minutes. Concentrated HCl was then added dropwise. A precipitate was formed, filtered and washed with water. 4-Methyl-5-phenylthiophen-3-amine (15 mg, 26% yield) was obtained as a light yellow oil.

1H NMR (400 MHz, CHLOROFORM-D) δ ppm 2.15 (s, 3 H) 6.20 (s, 1 H) 7.28-7.35 (m, 1 H) 7.37-7.46 (m, 4 H); ESI-MS: m/e═190.06 [M+H]⁺.

Step 4E: 4-Methyl-5-phenylthiophen-3-amine (15 mg, 0.079 mmol), methyl bromoacetate (15 μL, 0.158 mmol) and K₂CO₃ (44 mg, 0.316 mmol) in 2 mL DMF were stirred at 100° C. for 40 minutes. Water (10 mL) was then added, and the aqueous layer was extracted with 20 mL DCM. The organic layer was washed with water, brine and dried over Na₂SO₄. The crude product was purified by flash chromatography to give methyl 2-(4-methyl-5-phenylthiophen-3-ylamino)acetate (3 mg, 14% yield) as a light yellow oil.

1H NMR (400 MHz, CHLOROFORM-D) δ ppm 2.16 (s, 3 H) 3.81 (s, 3 H) 3.93 (s, 2 H) 5.90 (s, 1 H) 7.29-7.35 (m, 1 H) 7.37-7.45 (m, 4 H); ESI-MS: m/e=262.08 [M+H].⁺.

Step 4F: Methyl 2-((N-(tert-butoxycarbonyl)sulfamoyl)(4-methyl-5-phenylthiophen-3-yl)amino)acetate (16 mg, 26% yield) was prepared following the procedures in Step 1C of Example 1, using methyl 2-(4-methyl-5-phenylthiophen-3-ylamino)acetate (36 mg, 0.14 mmol), chlorosulfonyl isocyanate (36 μL, 0.42 mmol), tert-butanol (60 μL, 0.63 mmol) and diisopropylethylamine (122 μL, 0.7 mmol) as starting materials.

1H NMR (400 MHz, CHLOROFORM-D) δ ppm 1.49 (s, 9 H) 2.32 (s, 3 H) 3.73 (s, 3 H) 4.61 (s, 2 H) 7.30-7.36 (m, 1 H) 7.40-7.44 (m, 4 H) 7.50 (s, 1 H); ESI-MS: m/e=439.33 [M−H]⁻.

Step 4G: 5-(4-Methyl-5-phenyl-3-thienyl)-1,2,5-thiadiazolidin-3-one 1,1-dioxide was obtained as an offwhite solid (5.5 mg, 49% yield), following the procedure in Step 1D of Example 1 using methyl 2-((N-(tert-butoxycarbonyl)sulfamoyl)(4-methyl-5-phenylthiophen-3-yl)amino)acetate (16 mg, 0.036 mmol) as starting material.

1H NMP (400 MHz, CHLOROFORM-D) δ ppm 2.29 (s, 3 H) 4.42 (s, 2 H) 7.35-7.41 (m, 1 H) 7.42-7.46 (m, 4 H) 7.54 (s, 1 H); HRMS: calcd for C₁₃H₁₂N₂O₃S₂+H+, 309.03621; found (ESI-FTMS, [M+H]¹⁺), 309.0353.

### Example 5

### 5-[4-methyl-5-(3-nitrophenyl)-3-thienyll-1,2,5-thiadiazolidin-3-one 1,1-dioxide

Step 5A To a solution of methyl 4-methyl-3-(2,2,2-trifluoroacetamido)thiophene-2-carboxylate (534 mg, 2 mmol) in HOAc (4.0 mL) was added Br₂ (0.154 mL, 3.0 mmol) at room temperature. The reaction was allowed to stir for 4 h, and then quenched with aq. Na₂S₂O₃. The precipitate was collected by filtration and re-dissolved EtOAc, then washed with water. The organic layer was dried over Na₂SO₄ and concentrated in vacuo, affording methyl 5-bromo-4-methyl-3-(2,2,2-trifluoroacetamido)thiophene-2-carboxylate as a white solid (690 mg, 100%).

LCMS: m/e=344.14 [M+H]⁻

Step 5B: A reaction mixture of methyl 5-bromo-4-methyl-3-(2,2,2-trifluoroacetamido)thiophene-2-carboxylate (1.211 g, 3.5 mmol), Pd(PPh₃)₄ (400.75 mg, 0.35 mmol), 3-nitrophenylboronic acid (877.8 mg, 5.25 mmol) and KF (812 mg, 14 mmol) in THF (21 mL) was heated at 120° C. for 20 min. in microwave reactor. The reaction mixture was then diluted with EtOAc (50 mL) and washed with water. The organic layer was separated and the crude product was purified by flash column chromatography eluting with hexanes/EtOAc to give methyl 4-methyl-5-(3-nitrophenyl)-3-(2,2,2-trifluoroacetamido)thiophene-2-carboxylate (910 mg, 67%) as an off-white solid.

LCMS: m/e=387.27 [M+H]⁻

Step 5C: To a solution of methyl 4-methyl-5-(3-nitrophenyl)-3-(2,2,2-trifluoroacetamido)thiophene-2-carboxylate (567 mg, 1.5 mmol) in EtOH (60 mL) and water (6 mL) was added NaOH (360 mg, 9.0 mmol). The reaction was heated to 120° C. for 20 minutes under microwave conditions, after which it was judged complete by LCMS. The reaction mixture was transferred into an Erlenmeyer flask, and diluted with DMSO (10 mL). Concentrated HCl (10 mL) was added, and the mixture was allowed to stir at ambient temperature overnight. The reaction mixture was diluted with EtOAc, and neutralized with excess NaHCO₃(aq). Product was extracted into ethyl acetate (3×25 mL) and dried over MgSO₄, then concentrated in vacuo and purified by flash column chromatography eluting with hexane/EtOAc to afford 4-methyl-5-(3-nitrophenyl)thiophen-3-amine (110 mg, 31%) as a brown solid.

LCMS: m/e ═277.24 [M+H]⁻

Step 5D: To a solution of 4-methyl-5-(3-nitrophenyl)thiophen-3-amine (110 mg, 0.47 mmol) in DMF (3.0 mL) was added K₂CO₃ (85.6 mg, 0.94 mmol), followed by addition of methyl bromoacetate (0.047 mL, 0.52 mmol). The reaction was heated to 60° C. for 12 hours, then concentrated under vacuum. The crude solid was purified by flash chromatography eluted with hexane/ethyl acetate to give methyl 2-(4-methyl-5-(3-nitrophenyl)thiophen-3-ylamino)acetate (140 mg, 98%) as a yellow solid.

LCMS: m/e=307.25 [M+H]⁺

Step 5E: To a solution of chlorosulfonyl isocyanate (0.060 mL, 0.585 mmol) in DCM (3.0 mL) was added t-BuOH (0.074 mL, 0.693 mmol) dropwise. The solution was then stirred for 30 minutes to allow for complete formation of the desired tert-butoxycarbonylcarbamoylsulfamoyl chloride intermediate. The solution was then added to a solution of methyl 2-(4-methyl-5-(3-nitrophenyl)thiophen-3-ylamino)acetate (140 mg, 0.458 mmol) and Hunig's base (0.151 mL, 0.375 mmol) in DCM (2.0 mL) at 0° C. The reaction was allowed to stir at ambient temperature for an additional 2 hours. The solvent was removed under vacuum and the crude product was purified by flash chromatography eluting with hexane/EtOAc to give methyl 2-((N-(tert-butoxycarbonyl)sulfamoyl)(4-methyl-5-(3-nitrophenyl)thiophen-3-yl)amino)acetate (184.5 mg, 83%) as a yellow oily solid.

LCMS: m/e=484.34 [M+H]⁻

Step 5F: A solution of methyl 2-((N-(tert-butoxycarbonyl)sulfamoyl)(4-methyl-5-(3-nitrophenyl)thiophen-3-yl)amino)acetate (101 mg, 0.208 mmol) in DCM (4.0 mL) was cooled to 0° C. To the cooled solution was added TFA dropwise (1.0 mL). The reaction was allowed to stir at ambient temperature for 2 hours, then concentrated in vacuo, to afford the desired methyl 2-((4-methyl-5-(3-nitrophenyl)thiophen-3-yl)(sulfamoyl)amino)acetate (80 mg, 100%) as a yellow solid.

LCMS: m/e ═384.25 [M+H]⁻

Step 5G: To a solution of methyl 2-((4-methyl-5-(3-nitrophenyl)thiophen-3-yl)(sulfamoyl)amino)acetate (80 mg, 0.2 mmol) in anhydrous THF (2.0 mL) cooled to 0° C. was added a slurry of NaH (60% in mineral oil, 24 mg, 0.6 mmol). The solution was then stirred for 2 hours at ambient temperature, then diluted with H₂O (4 mL). The aqueous solution was washed with hexane (2×5 mL) and then acidified via drop-wise addition of 10% HCl, precipitating the desired cyclized final product 5-[4-methyl-5-(3-nitrophenyl)-3-thienyl]-1,2,5-thiadiazolidin-3-one 1,1-dioxide (68 mg, 96%) as a light brown solid. LCMS: m/e=352.22 [M−H]⁻.

### Example 6

### 4-methyl-5-(5-phenyl-3-thienyl)-1,2,5-thiadiazolidin-3-one 1,1-dioxide

Step SD: To a solution of 5-phenyl-thiophen-3-amine (175 mg, 1.0 mmol) in DMF (5.0 mL) was added K₂CO₃ (276 mg, 2.0 mmol), followed by addition of methyl 2-methyl-bromoacetate (0.139 mL, 1.2 mmol). The reaction was heated to 60° C. for 12 hours, then concentrated under vacuum. The crude solid was purified by flash chromatography eluted with hexane/ethyl acetate to give methyl 2-(5-phenylthiophen-3-ylamino)propanoate (210 mg, 80%) as a light yellow oil.

LCMS: m/e ═262.21 [M+H]⁺

Step 5E: To a solution of chloro-sulfonyl isocyanate (0.1 mL, 1.15 mmol) in DCM (5.0 mL) was added t-BuOH (0.146 mL, 1.153 mmol) dropwise. The solution was then stirred for 30 minutes to allow for complete formation of the desired tert-butoxycarbonylcarbamoylsulfamoyl chloride intermediate. The solution was then added to a solution of methyl 2-(5-phenylthiophen-3-ylamino)propanoate (200 mg, 0.766 mmol) and Hunig's base (0.397 mL, 2.3 mmol) in DCM (2.0 mL) at 0° C. The reaction was allowed to stir at ambient temperature for an additional 2 hours. The solvent was removed under vacuum and the crude product was purified on SiO2 column eluted with hexanes/EtOAc to give methyl 2-((N-(tert-butoxycarbonyl)sulfamoyl)(5-phenylthiophen-3-yl)amino)propanoate (260 mg, 77%) as a yellow oily solid.

LCMS: m/e ═439.25 [M+H]⁻

Step 5F: A solution of methyl 2-((N-(tert-butoxycarbonyl)sulfamoyl)(5-phenylthiophen-3-yl)amino)propanoate (250 mg, 0.568 mmol) in DCM (5.0 mL) was cooled to 0° C. To the cooled solution was added TFA dropwise (2.0 mL). The reaction was allowed to stir at ambient temperature for 2 hours, then concentrated in vacuo, to afford the desired methyl 2-((5-phenylthiophen-3-yl)(sulfamoyl)amino)propanoate, which was re-dissolved in THF (5 mL). To this was added NaH (60% in mineral oil, 80 mg, 2 mmol). The resultant mixture was stirred at room temperature for 2 hours, then diluted with water (20 mL) and acidified with conc. HCl. The desired product, 4-methyl-5-(5-phenyl-3-thienyl)-1,2,5-thiadiazolidin-3-one 1,1-dioxide (110 mg, 63% overall), was obtained as a dark brown solid.

HRMS: calcd for C₁₃H₁₂N₂O₃S₂+H+, 309.03621; found (ESI-FTMS, [M+H]¹⁺), 309.366.

### Example 7

### 5-(4-methyl-5-{3-[(3,3,5,6,-tetramethylcyclohexyl)amino]phenyl}-3-thienyl]-1,2,5-thiadiazolidin-3-one 1,1-dioxide

Step 5F: To a solution of 2-((N-(tert-butoxycarbonyl)sulfamoyl)(4-methyl-5-(3-nitrophenyl)thiophen-3-yl)amino)acetate (180 mg, 0.37 mmol), obtained from Step 5E in Example 5, in EtOAc (10 mL) and MeOH (10 mL) was added Pd/C (˜20 mg) under N₂. To this solution was applied a hydrogen balloon. The resultant mixture was stirred at room temperature for 2 hour, then filtered through a pad of Celite. The solvent was removed under vacuum to give the desired methyl 2-((5-(3-aminophenyl)-4-methylthiophen-3-yl)(N-(tert-butoxycarbonyl)sulfamoyl)amino)acetate (165 mg, 98%) as a light yellow solid.

1H NMR (400 MHz, CHLOROFORM-D) δ ppm 1.43 (s, 9 H) 2.19 (s, 3 H) 3.68 (s, 3 H) 4.46 (s, 2 H) 6.54-6.62 (m, 1 H) 6.62-6.69 (m, 1 H) 6.71-6.78 (m, 1 H) 7.11 (t, J=7.71 Hz, 1 H) 7.42 (s, 1 H)

Step 5G: To a solution of methyl 2-((5-(3-aminophenyl)-4-methylthiophen-3-yl)(N-(tert-butoxycarbonyl)sulfamoyl)amino)acetate (55 mg, 0.12 mmol) in MeOH (2 mL) was added 1,1,3,3-tetramethylcyclohexanone (31.2 μL, 0.18 mmol), HOAc (10.6 μL, 0.18 mmol) and then NaBH₃CN (51 mg, 0.24 mmol) at room temperature. The resultant mixture was stirred at room temperature overnight. The solvent was removed in vacuum. The crude mixture was purified flash chromatography eluted with hexane/EtOAc to give the desired product (55 mg, 77%), methyl 2-((N-(tert-butoxycarbonyl)sulfamoyl)(4-methyl-5-(3-(3,3,5,5-tetramethylcyclohexylamino)phenyl)thiophen-3-yl)amino)acetate, as a light yellow oil.

1H NMR (400 MHz, CHLOROFORM-D) δ ppm 1.51 (s, 9 H) 1.68-1.79 (m, 1 H) 1.82-1.92 (m, 2 H) 2.16 (s, 12 H) 2.29 (s, 3 H) 3.53-3.67 (m, 1 H) 3.76 (s, 3 H) 4.54 (s, 2 H) 6.56 (dd, J=7.71, 1.89 Hz, 1 H) 6.61-6.67 (m, 1 H) 6.73 (d, J=8.08 Hz, 1 H) 7.19 (t, J=7.83 Hz, 1 H) 7.50 (s, 1 H)

Step 5H: To a solution of methyl 2-((N-(tert-butoxycarbonyl)sulfamoyl)(4-methyl-5-(3-(3,3,5,5-tetramethylcyclohexylamino)phenyl)thiophen-3-yl)amino)acetate (55 mg, 0.093 mmol) in DCM (5 mL) was added TFA (1 mL). The resultant mixture was stirred at room temperature for 4 hour. The solvent was removed under vacuum to give the desired product, methyl 2-((4-methyl-5-(3-(3,3,5,5-tetramethylcyclohexylamino)phenyl)thiophen-3-yl)(sulfamoyl)amino)acetate, used in the next step without further purification.

LCMS: m/e=494.46 [M+H]⁺

Step 5I: To a solution of methyl 2-((4-methyl-5-(3-(3,3,5,5-tetramethylcyclohexylamino)phenyl)thiophen-3-yl)(sulfamoyl)amino)acetate (˜0.093 mol) in THF was added NaH (60% in mineral oil, 40 mg) at 0° C. The resultant mixture was stirred at room temperature for 2 hour before acidified with 1N aq. HCl and then diluted with water (10 mL). The desired product, 5-(4-methyl-5-{3-[(3,3,5,6,-tetramethylcyclohexyl)amino]phenyl}-3-thienyl]-1,2,5-thiadiazolidin-3-one 1,1-dioxide, was collected by filtration as a light yellow solid (32 mg, 75%).

HRMS: calcd for C₂₃H₃₁N₃O₃S₂+H+, 462.18796; found (ESI-FTMS, [M+H]¹⁺), 462.1886.

### Example 8

### 5-5-[3-(cyclohexylamino)phenyl]-3-thienyl}-1,2,5-thiadiazolidin-3-one 1,1-dioxide

Step 5G: To a solution of methyl 2-((5-(3-aminophenyl)-4-methylthiophen-3-yl)(N-(tert-butoxycarbonyl)sulfamoyl)amino)acetate (55 mg, 0.12 mmol) in MeOH (2 mL), was added cyclohexanone ((18.7 μL, 0.18 mmol), HOAc (10.6 μL, 0.18 mmol) and then NaBH₃CN (51 mg, 0.24 mmol) at room temperature. The resultant mixture was stirred at room temperature overnight. The solvent was removed in vacuum. The crude mixture was purified by flash chromatography eluted with hexane/EtOAc to give the desired product (45 mg, 70%), methyl 2-((N-(tert-butoxycarbonyl)sulfamoyl)(5-(3-(cyclohexylamino)phenyl)-4-methylthiophen-3-yl)amino)acetate, as a light yellow oil.

1H NMR (400 MHz, CHLOROFORM-D) δ ppm 0.97-1.38 (m, 4 H) 1.43 (s, 9 H) 1.45-2.07 (m, 6 H) 2.19 (s, 3 H) 3.05-3.26 (m, 1 H) 3.69 (s, 3 H) 4.46 (s, 2 H) 6.49 (dd, J=7.83, 2.02 Hz, 1 H) 6.55 (t, J=1.89 Hz, 1 H) 6.64 (d, J=8.08 Hz, 1 H) 7.10 (t, J=7.96 Hz, 1 H) 7.42 (s, 1 H)

Step 5H: To a solution of methyl 2-((N-(tert-butoxycarbonyl)sulfamoyl)(5-(3-(cyclohexylamino)phenyl)-4-methylthiophen-3-yl)amino)acetate (45 mg, 0.084 mmol) in DCM (5 mL) was added TFA (1 mL). The resultant mixture was stirred at room temperature for 4 hour. The solvent was removed under vacuum to give the desired product, methyl 2-((5-(3-(cyclohexylamino)phenyl)-4-methylthiophen-3-yl)(sulfamoyl)amino)acetate, used in the next step without further purification.

LCMS: m/e ═438.35 [M+H]⁺

Step 5I: To a solution of methyl 2-((5-(3-(cyclohexylamino)phenyl)-4-methylthiophen-3-yl)(sulfamoyl)amino)acetate (˜0.084 mol) in THF was added NaH (60% in mineral oil, 40 mg) at 0° C. The resultant mixture was stirred at room temperature for 2 hour before acidified with 1N aq. HCl and then diluted with water (10 mL). The desired product, 5-{5-[3-(cyclohexylamino)phenyl]-3-thienyl}-1,2,5-thiadiazolidin-3-one 1,1-dioxide, was collected by filtration as a light yellow solid (15 mg, 44%).

HRMS: calcd for Cl₉H₂₃N₃O₃S₂+H+, 406.12536; found (ESI-F[MS, [M+H]¹⁺), 406.1258

### Example 9

### 5-[5-(3-{[1-(benzylsulfonyl)piperidine-4-yl]amino}phenyl)-4-methyl-3-thienyl]-1,2,5-thiadiazolidin-3-one 1,1-dioxide

Step 6A: A reaction mixture of methyl 5-bromo-4-methyl-3-(2,2,2-trifluoroacetamido)thiophene-2-carboxylate (890 mg, 2.57 mmol), Pd(PPh₃)₄ (286 mg, 0.25 mmol), 3-aminophenylboronic acid (598 mg, 3.86 mmol) and KF (596 mg, 10.28 mmol) in THF (10 mL) was heated at 120° C. for 20 min in microwave reactor. The reaction mixture was then diluted with EtOAc (50 mL) and washed with aq. NH₄Cl. The organic layer was separated and the crude product was purified on SiO₂ gel flash column eluted with hexane/EtOAc to give methyl methyl 5-(3-aminophenyl)-4-methyl-3-(2,2,2-trifluoroacetamido)thiophene-2-carboxylate (720 mg, 78%) as an off-white solid.

Step 6B: To a solution of methyl 2-((5-(3-aminophenyl)-4-methylthiophen-3-yl)(N-(tert-butoxycarbonyl)sulfamoyl)amino)acetate (304 mg, 0.85 mmol) in MeOH (4 mL) was added 1-(benzylsulfonyl)piperidin-4-one ((255 mg, 1.02 mmol), HOAc (60.5 μL, 1.02 mmol) and then NaBH₃CN (62.84 mg, 1.0 mmol) at room temperature. The resultant mixture was stirred at room temperature overnight. The solvent was removed in vacuum. The crude mixture was purified on SiO₂ column eluted with hexane/EtOAc to give the desired product (180 mg, 36%), methyl 5-(3-(1-(benzylsulfonyl)piperidin-4-ylamino)phenyl)-4-methyl-3-(2,2,2-trifluoroacetamido)thiophene-2-carboxylate, as a light yellow oil.

LCMS: m/e=594.57 [M+H]⁻

Step 6C: To a solution of methyl 5-(3-(1-(benzylsulfonyl)piperidin-4-ylamino)phenyl)-4-methyl-3-(2,2,2-trifluoroacetamido)thiophene-2-carboxylate (150 mg, 0.252 mmol) in EtOH (4 mL) and water (2 mL) was added NaOH (600 mg, 15.0 mmol). The reaction was heated to 60° C. for 2 hours, after which it was judged complete by LCMS. Concentrated HCl (10 mL) was added, and the mixture was allowed to stir for 30 minutes, and then basified with aq. NH₃. The reaction mixture was diluted with DCM, the organic layer was separated, then concentrated in vacuo and purified flash chromatography eluted with hexane/EtOAc, affording N-(3-(4-amino-3-methylthiophen-2-yl)phenyl)-1-(benzylsulfonyl)piperidin-4-amine (110 mg, 99%) as a brown solid.

LCMS: m/e=442.38 [M+H]⁺

Step 6D: To a solution of N-(3-(4-amino-3-methylthiophen-2-yl)phenyl)-1-(benzylsulfonyl)piperidin-4-amine (145 mg, 0.329 mmol) in DMF (3.0 mL) was added K₂CO₃ (90.8 mg, 0.658 mmol), followed by addition of methyl bromoacetate (0.036 mL, 0.394 mmol). The reaction was stirred at room temperature for 2 days, then concentrated under vacuum. The crude solid was purified by flash chromatography eluted with hexane/ethyl acetate to give methyl 2-(5-(3-(1-(benzylsulfonyl)piperidin-4-ylamino)phenyl)-4-methylthiophen-3-ylamino)acetate (121 mg, 72%) as a yellow solid.

LCMS: m/e ═514.42 [M+H]⁺

Step 6E: To a solution of methyl 2-(5-(3-(1-(benzylsulfonyl)piperidin-4-ylamino)phenyl)-4-methylthiophen-3-ylamino)acetate in DCM (2 mL) was added tert-butoxycarbonylcarbamoylsulfamoyl chloride (˜1.0 M DCM solution, 0.234 mL, 0.234 mmol) and DIPEA (67.9 μL, 0.39 mmol) dropwise. The reaction was allowed to stir at ambient temperature for an additional 2 hours. The solvent was removed under vacuum and the crude product was purified by flash chromatography eluted with hexane/EtOAc to give methyl 2-((5-(3-(1-(1-(benzylsulfonyl)piperidin-4-yl)-3-(tert-butoxycarbonyl)ureido)phenyl)-4-methylthiophen-3-yl)(N-(tert-butoxycarbonyl)sulfamoyl)amino)acetate (96 mg, 89%) as a yellow oily solid.

LCMS: m/e ═838.47 [M+H]⁻

Step 6F: To a solution of methyl 2-((5-(3-(1-(1-(benzylsulfonyl)piperidin-4-yl)-3-(tert-butoxycarbonyl)ureido)phenyl)-4-methylthiophen-3-yl)(N-(tert-butoxycarbonyl)sulfamoyl)amino)acetate (60 mg, 0.0867 mmol) in DCM (4.0mL) was added TFA dropwise (1.0 mL). The reaction was allowed to stir at ambient temperature for 2 hours, then concentrated in vacuo, to afford crude methyl 2-((5-(3-(1-(1-(benzylsulfonyl)piperidin-4-yl)ureido)phenyl)-4-methylthiophen-3-yl)(sulfamoyl)amino)acetate, which was then dissolved in THF (2 mL). To this was added NaH (60% in mineral oil, 40 mg) at 0° C. The reaction mixture was allowed to stir at room temperature for 2 hours before diluted with MeOH (2 mL) and 2N NaOH (2 mL). The resultant mixture was stirred at 60° C. overnight before acidified with IN HCl. The desired product, 5-[5-(3-{[1-(benzylsulfonyl)piperidine-4-yl]amino}phenyl)-4-methyl-3-thienyl]-1,2,5-thiadiazolidin-3-one 1,1-dioxide, was collected by filtration as a light yellow solid (18 mg, 37% overall).

HRMS: calcd for C₂₅H₂₈N₄O₅S₃+H+, 561.12946; found (ESI-FFMS, [M+H]¹⁺), 561.1292.

### Example 10

### N-{3-[3-chloro-4-(1,1-dioxido-4-oxo-1,2,5-thiadiazolidin-2-yl)-2-thienyl]phenyl}-N-cyclohexylsulfamide

Step 7A: A reaction mixture of methyl 3-acetamido-4,5-dichlorothiophene-2-carboxylate (1.59 g, 6 mmol), Pd(PPh₃)₄ (687 mg, 0.6 mmol), 3-aminophenylboronic acid (1.398 g, 9.0 mmol) and KF (1.392 g, 24 mmol) in THF (24 mL) was heated at 120° C. for 25 min. in microwave reactor. The reaction mixture was then diluted with EtOAc (50 mL) and washed with aq. NH₄Cl. The organic layer was separated and the crude product was purified flash chromatography eluted with hexane/EtOAc to give methyl 3-acetamido-5-(3-aminophenyl)-4-chlorothiophene-2-carboxylate (1.8 g, 92%) as an off-white solid.

1H NMR (400 MHz, CHLOROFORM-D) δ ppm 2.25 (s, 3 H) 3.79 (s, 1 H) 3.89 (s, 3 H) 6.71-6.77 (m, 1 H) 6.98-7.02 (m, 1 H) 7.04 (dd, J=6.44, 1.39 Hz, 1 H) 7.23 (t, J=7.83 Hz, 1 H)

Step 7B: To a solution of methyl 3-acetamido-5-(3-aminophenyl)-4-chlorothiophene-2-carboxylate (648 mg, 2.0 mmol) in MeOH (8 mL) was added 1-cyclohexanone ((0.571 mL, 6.0 mmol), HOAc (177 μL, 3.0 mmol) and then NaBH₃CN (125.7 mg, 2.0 mmol) at room temperature. The resultant mixture was stirred at room temperature for 1 h. The solvent was removed in vacuum. The crude mixture was purified on SiO₂ column eluted with hexane/EtOAc to give the desired product (795 mg, 98%), methyl 3-acetamido-4-chloro-5-(3-(cyclohexylamino)phenyl)thiophene-2-carboxylate, as a light yellow oil.

Step 7C: To a solution of methyl 3-acetamido-4-chloro-5-(3-(cyclohexylamino)phenyl)thiophene-2-carboxylate (406 mg, 1.0 mmol) in EtOH (4 mL) and water (2 mL) was added NaOH (200 mg, 5.0 mmol). The reaction was heated to 120° C. for 50 minutes in microwave oven, after which it was judged complete by LCMS. Concentrated HCl (10 mL) was added, and the mixture was allowed to stir at 60° C. for 2 hours, and then basified with aq. NH₃. The reaction mixture was diluted with DCM, the organic layer was separated, then concentrated in vacuo and purified on SiO₂ gel column eluted with hexane/EtOAc, affording 4-chloro-5-(3-(cyclohexylamino)phenyl)thiophen-3-amine (1.28 g, 91%) as a brown solid.

LCMS: m/e ═393.36 [M+H]⁺

Step 7D: To a solution of 4-chloro-5-(3-(cyclohexylamino)phenyl)thiophen-3-amine (290 mg, 0.948 mmol) in DMF (4.0 mL) was added K₂CO₃ (196.2 mg, 1.422 mmol), followed by addition of methyl bromoacetate (0.096 mL, 1.04 mmol). The reaction was stirred at room temperature for one day, then concentrated under vacuum. The crude solid was purified by flash chromatography eluted with hexane/ethyl acetate to give methyl 2-(4-chloro-5-(3-(cyclohexylamino)phenyl)thiophen-3-ylamino)acetate (305 mg, 85%) as a yellow oil.

LCMS: m/e ═379.30 [M+H]⁺

Step 7E: To a solution of methyl 2-(4-chloro-5-(3-(cyclohexylamino)phenyl)thiophen-3-ylamino)acetate (216 mg, 0.571 mmol) in DCM (3 mL) was added tert-butoxycarbonylcarbamoylsulfamoyl chloride (˜1.0 M DCM solution, 0.86 mL, 0.86 mmol) and DIPEA (248.6 μL, 1.43 mmol) dropwise. The reaction was allowed to stir at ambient temperature for an additional 2 hours. The solvent was removed under vacuum and the crude product was purified by flash chromatography eluted with hexane/EtOAc to give methyl 2-((N-(tert-butoxycarbonyl)sulfamoyl)(5-(3-((N-(tert-butoxycarbonyl)sulfamoyl)(cyclohexyl)amino)phenyl)-4-chlorothiophen-3-yl)amino)acetate (310 mg, 78%) as a yellow oily solid.

LCMS: m/e ═558.36 [M+H]⁺

Step 7F: To a solution of methyl 2-((N-(tert-butoxycarbonyl)sulfamoyl)(5-(3-((N-(tert-butoxycarbonyl)sulfamoyl)(cyclohexyl)amino)phenyl)-4-chlorothiophen-3-yl)amino)acetate (150 mg, 0.214 mmol) in DCM (5.0 mL) was added TFA dropwise (2.0 mL). The reaction was allowed to stir at ambient temperature for 2 hours, then concentrated in vacuo, to afford crude methyl 2-((4-chloro-5-(3-(1-cyclohexylureido)phenyl)thiophen-3-yl)(sulfamoyl)amino)acetate, which was then dissolved in THF (4 mL). To this was added NaH (60% in mineral oil, 40 mg) at 0 ° C. The reaction mixture was allowed to stir at room temperature for 2 hours before acidified with 1N HCl. The desired product, N-{3-[3-chloro-4-(1,1-dioxido-4-oxo-1,2,5-thiadiazolidin-2-yl)-2-thienyl]phenyl}-N-cyclohexylsulfamide (58 mg, 54%), was collected as a light yellow solid.

LCMS m/e 456.36 [M]⁻

### Example 11

### 5-{4-chloro-5-[3-(cyclohexylamino)phenyl]-3-thienyl}-1,2,5-thiadiazolidin-3-one 1,1-dioxide

Step 7G: A solution of N-{3-[3-chloro-4-(1,1-dioxido-4-oxo-1,2,5-thiadiazolidin-2-yl)-2-thienyl]phenyl}-N-cyclohexylsulfamide in EtOH (2 mL) and 2N NaOH (2 mL) was heated at 60° C. overnight before acidified with 1N HCl. The desired product, -14-chloro-5-[3-(cyclohexylamino)phenyl]-3-thienyl}-1,2,5-thiadiazolidin-3-one 1,1-dioxide, was collected by filtration as a light yellow solid (18 mg, 74%).

HRMS: calcd for C₁₈H₂₀ClN₃O₃S₂+H+, 426.07074; found (ESI-FTMS, [M+H]¹⁺), 426.0708.

### Example 12

### 5-(4-bromo-5-phenyl-3-thienyl)-1,2,5-thiadiazolidin-3-one 1,1-dioxide

Step 8A: To a solution of methyl-3-amino-5-phenyl thiophene-2-carboxylate (1.0 g, 4.10 mmol) in HOAc (15.0 mL) was added Br₂ (0.63 mL, 12.3 mmol). The reaction was heated to 60° C. and allowed to stir approximately 45 minutes, after which it was judged complete by LCMS. The excess Br₂ was quenched with excess Na₂S₂O₃, and the crude product was extracted (3×25 mL) into DCM. The combined organic layers were dried over MgSO₄ and concentrated in vacuo, affording 1.6 g of crude methyl 3-amino-4-bromo-5-phenylthiophene-2-carboxylate as a light yellow solid >90% purity.

1H NMR (400 MHz, CHLOROFORM-D) δ ppm 3.87 (s, 3 H) 5.74 (br. s, 2 H) 7.41-7.49 (m, 3 H) 7.63-7.68 (m, 2 H); LCMS: m/e=282.1 [M+H]⁺

Step 8B: To a solution of methyl 3-amino-4-bromo-5-phenylthiophene-2-carboxylate (˜4.10 mmol) in EtOH (40.0 mL) was added 15% NaOH(aq) (6.8 mL, 25.7 mmol). The reaction was heated to 100° C. for 30 minutes under microwave conditions, after which it was judged complete by LCMS. The reaction mixture was transferred into an Erlenmeyer flask, and diluted with excess EtOH (10.0 mL). Concentrated HCl (8.0 mL), and allowed to stir at ambient temperature for one hour, after which the decarboxylation was judged complete by LCMS. The reaction mixture was diluted with H₂O, and neutralized with excess NaHCO₃(aq). Product was extracted into ethyl acetate (3×25 mL) and dried over MgSO₄, then concentrated in vacuo, affording 1.05 g of 4-bromo-5-phenylthiophen-3-amine in >90% purity. No purification was necessary. (>99% Yield)

1H NMR (400 MHz, CHLOROFORM-D) δ ppm 3.86 (br. s, 2 H) 6.28 (s, 3 H) 7.32-7.48 (m, 3 H) 7.58-7.68 (m, 2 H);

LCMS: m/e=254.1 [M+H]⁺

Step 8C: A solution of 4-bromo-5-phenylthiophen-3-amine (500 mg, 2.0 mmol) in DMF (10.0 mL) was cooled to 0° C. To the cooled solution was added K₂CO₃ (680 mg, 4.9 mmol), followed by addition of methyl bromoacetate (0.2 mL, 0.22 mmol). The reaction was heated to 120° C. for 20 minutes under microwave conditions, after which it was judged complete by LCMS. The reaction mixture was diluted with H₂O, and the product was extracted into ethyl acetate (3×25 mL) and dried over MgSO₄, then concentrated in vacuo, to afford the desired methyl 2-(4-bromo-5-phenylthiophen-3-ylamino)acetate. The crude solid was purified by flash chromatography, using a 2%-20% ethyl acetate/hexane solvent gradient. (400 mg; 62%)

1H NMR (400 MHz, CHLOROFORM-D) δ ppm 3.81 (s, 3 H) 3.94 (d, J=5.31 Hz, 2 H) 4.58 (br. s, 1 H) 5.94 (s, 1 H) 7.31-7.47 (m, 3 H) 7.56-7.70 (m, 2 H);

LCMS: m/e=325.0 [M+H]⁺

Step 8D: To a solution of chlorosulfonyl isocyanate (0.12 mL, 1.4 mmol) in DCM (3.0 mL) was added t-BuOH (0.2 mL, 2.1 mmol) dropwise. The solution was then stirred for 30 minutes to allow for complete formation of the desired tert-butoxycarbonylcarbamoylsulfamoyl chloride intermediate. The solution was then added to a solution of methyl 2-(4-bromo-5-phenylthiophen-3-ylamino)acetate (400 mg, 1.2 mmol) and Hunig's base (0.31 mL, 1.8 mmol) in DCM (10.0 mL). The reaction was allowed to stir at ambient temperature for an additional 30 minutes, after which it was judged complete by LCMS. The reaction mixture was diluted with DCM, washed with 5% HCl (2×10 mL), and dried over MgSO₄, then concentrated in vacuo, to afford the desired methyl 2-((4-bromo-5-phenylthiophen-3-yl)(N-(tert-butoxycarbonyl)sulfamoyl)amino)acetate. The resultant crude oil was purified by flash chromatography, using a 2%-20% ethyl acetate/hexanes solvent gradient. (230 mg; 38%).

1H NMR (400 MHz, CHLOROFORM-D) δ ppm 7.30-7.35 (m, 1 H) 7.38-7.49 (m, 3 H) 7.61 (dd, J=8.08, 1.52 Hz, 2 H) 7.86 (br. s, 1 H); LCMS: m/e=504.0 [M+H]⁺

Step 8E: A solution of methyl 2-((4-bromo-5-phenylthiophen-3-yl)(N-(tert-butoxycarbonyl)sulfamoyl)amino)acetate (230 mg, 0.6 mmol) in DCM (5.0 mL) was cooled to 0° C. To the cooled solution was added TFA dropwise (2.0 mL, 40% vol). The reaction was allowed to stir at ambient temperature for 1.5 hour, after which it was judged complete by LCMS. The reaction mixture then concentrated in vacuo, to afford the desired methyl 2-((4-bromo-5-phenylthiophen-3-yl)(sulfamoyl)amino)acetate as a TFA salt in >95% purity. The resultant crude solid was carried on to the final step without purification. (220 mg; >99%).

LCMS: m/e=404.0 [M+H]⁺

Step 8F: To a solution of methyl 2-((4-bromo-5-phenylthiophen-3-yl)(sulfamoyl)amino)acetate (˜0.46 mmol) in anhydrous THF (3.0 mL) cooled to 0° C. was added a slurry of NaH (60% in mineral oil) in THF (55mg/2mL) dropwise. The solution was then stirred for 30 minutes at ambient temperature after which it was judged complete by LCMS. The reaction mixture was diluted with H₂O (1 mL), and basified to pH ˜12 with 1N NaOH. The aqueous solution was washed with hexanes (2×10 mL) and then acidified via drop-wise addition of 10% HCl, precipitating the desired cyclized final product 5-{4-chloro-5-[3-(cyclohexylamino)phenyl]-3-thienyl }-1,2,5-thiadiazolidin-3-one 1,1-dioxide (125 mg; 71%).

1HNMR (400 MHz, DMSO-D6) δ ppm 4.39 (s, 2 H) 7.40-7.57 (m, 3 H) 7.57-7.71 (m, 2 H) 7.94 (s, 1H); LCMS: m/e=371.9 [M+H]⁺

### Example 13

### Biological Testing

Evaluation of the utility of a PTPase inhibitor, such as those described here, can be performed using a variety of methods previously described, with generally applicable techniques and specific examples (1-5).

1. Fersht, A. Structure and Mechanism in Protein Science: A Guide to Enzyme Catalysis and Protein Folding (W.H. Freeman and Company, New York, 1999).

2. McCain D F, Zhang Z Y: Assays for protein-tyrosine phosphatases. Methods Enzymol. (2002) 345:507-518.

3. Tonks N K, Diltz C D, Fisher E H: Characterization of the major protein tyrosine phophatases of human placenta. J. Biol. Chem. (1988) 263:6731-6737.

4. Barford D, Flint A J, Tonks N K: Crystal structure of human protein tyrosine phosphatase 1B. Science (1994) 263:1397-1404.

5. Huyer G, Lui S, Kelly J, Moffat J, Payette P, Kennedy B, Tsaprailus G, Gresser M J, Ramachandran C: Mechanism of inhibition of protein-tyrosine phosphatases by vanadate and pervanandate. J. Biol. Chem. (1997) 272:843-851.”

The source of PTP1b activity is the catalytic domain of human PTP1b (residues 1-299) expressed and purified from E coli. The p-nitrophenyl phosphate (pNPP) substrate was purchased from Sigma. PTPase activity was measured at room temperature (25° C.) in a 200 ul reaction mixture with various concentrations of substrate. Reactions began with enzyme addition and were quenched after 2-3 minutes with 1N NaOH. Background hydrolysis of pNPP was measured without addition of enzyme. Cleaved p-nitrophenyl produced was determined by measuring the absorbance at 405 nm using a molar extinction coefficient of 18,000 M−1 cm−1. All experiments were performed in 50 mM 3,3-dimethylglutarate buffer (pH 7.0), with 10 mM DTT, 1 mM EDTA, and with the ionic strength adjusted to 0.15 M with NaCl. All solutions were prepared in distilled, deionized water. For initial evaluation of each compound, percent inhibition was determined for two concentrations of compound (for example at 200 uM and 20 uM) at a fixed pNPP concentration. Inhibition constants (Ki's) for each active compound were then determined by measuring the initial rate of hydrolysis of various substrate concentrations at three different fixed concentrations of each inhibitor.

Percent inhibition was determined following background subtraction of the non-enzymatic hydrolysis rate compared to no inhibitor (DMSO only) controls. Inhibition constants and inhibition patterns were evaluated using the direct curve-fitting program GraFit (Erithacus Software Limited).

Inhibition constants for the compounds of Examples 1-12 are provided in the table below.

| | Kᵢ |
|---|---|
| | Micromolar |
| Compound Name | (uM) |
| 5-(5-phenyl-3-thienyl)-1,2,5-thiadiazolidin-3-one 1,1-dioxide | 150 |
| 5-(4-chloro-5-phenyl-3-thienyl)-1,2,5-thiadiazolidin-3-one 1,1-dioxide | 16 |
| 5-(2,4-dichloro-5-phenyl-3-thienyl)-1,2,5-thiadiazolidin-3-one 1,1- | 16 |
| dioxide | |
| 5-(4-methyl-5-phenyl-3-thienyl)-1,2,5-thiadiazolidin-3-one 1,1-dioxide | 41 |
| 5-[4-methyl-5-(3-nitrophenyl)-3-thienyl]-1,2,5-thiadiazolidin-3-one 1,1- | 34 |
| dioxide | |
| 4-methyl-5-(5-phenyl-3-thienyl)-1,2,5-thiadiazolidin-3-one 1,1-dioxide | >1000 |
| 5-(4-methyl-5-{3-[(3,3,5,6,-tetramethylcyclohexyl)amino]phenyl}-3- | 15 |
| thienyl]-1,2,5-thiadiazolidin-3-one 1,1-dioxide | |
| 5-{5-[3-(cyclohexylamino)phenyl]-3-thienyl}-1,2,5-thiadiazolidin-3-one 1,1- | 12 |
| dioxide | |
| 5-[5-(3-{[1-(benzylsulfonyl)piperidine-4-yl]amino}phenyl)4-methyl-3- | 4 |
| thienyl]-1,2,5-thiadiazolidin-3-one 1,1-dioxide | |
| N-{3-[3-chloro-4-(1,1-dioxido-4-oxo-1,2,5-thiadiazolidin-2-yl)-2- | 1.7 |
| thienyl]phenyl}-N-cyclohexylsulfamide | |
| 5-{4-chloro-5-[3-(cyclohexylamino)phenyl]-3-thienyl}-1,2,5- | 14 |
| thiadiazolidin-3-one 1,1-dioxide | |
| 5-(4-bromo-5-phenyl-3-thienyl)-1,2,5-thiadiazolidin-3-one 1,1-dioxide | 18 |

### REFERENCES

1) Saltiel, A. R. “New Perspectives into the Molecular Pathogenesis and Treatment of Type 2 Diabetes” Cell 104: 517-529 (2001).

2) Elchebly, et al. “Increased Insulin Sensitivity and Obesity Resistance in Mice Lacking the Protein Tyrosine Phosphatase-1B Gene” Science 283:1544-1548 (1999).

3) Klaman, et al. “Increased energy Expenditure, Decreased Adiposity, and Tissue-Specific Insulin Sensitivity in Protein-Tyrosine Phosphatase 1B-Deficient Mice” Mol. Cell. Biol. 20: 5479-5489 (2000).

4) Zhang, Y. L., Zhang, Z. Y. “Low-affinity Binding Determined by Titration Calorimetry Using a High-affinity Coupling Ligand: A Thermodynamic Study of Ligand Binding to Protein Tyrosine Phosphatase 1B” Anal. Biochem. 261: 139-148 (1998).

5) Zhang, et al. “Thermodynamic Study of Ligand Binding to Protein-Tyrosine Phosphatase 1B and Its Substrate-Trapping Mutants” J. Biol. Chem. 275: 34205-34212 (2000).

6) Zhang, et al. “Dissecting the Catalytic Mechanism of Protein-Tyrosine Phosphatases” Proc. Natl. Acad. Sci. USA 91: 1624-1627 (1994).

7) Iversen, et al. “Structure-based Design of a Low Molecular Weight, Nonphosphorus, Nonpeptide, adn Highly Selective Inhibitor of Protein-tyrosine Phosphatase 1B” J. Biol. Chem. 275: 10300-10307 (2000).

8) Sarmiento, et al. “Structure-Based Discovery of Small Molecule Inhibitors Targeted to Protein Tyrosine Phosphatase 1B,” J. Med. Chem. 43: 146-155 (2000).

9) Taing, et al. “Potent and Highly Selective Inhibitors of the Protein Tyrosine Phosphatase 1B,” Biochemistry 38: 3793-3803 (1999).

A number of embodiments of the invention have been described. Nevertheless, it will be understood that various modifications may be made without departing from the spirit and scope of the invention. Accordingly, other embodiments are in the claims.

What is claimed is:

1. A compound of formula (IV): 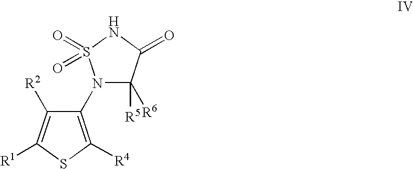
wherein
R¹is C₆-C₁₆ aryl or heteroaryl including 5-16 atoms, each of which is optionally substituted with from 1-10 R^{a};
R² and R are each, independently:
(i) hydrogen; or
(ii) halo; NR^{b}R^{c}; nitro; azido; hydroxy; C₁-C₁₂ alkoxy or C₁-C₁₂ thioalkoxy, each of which is optionally substituted with 1-5 R^{d}; C₁-C₁₂ haloalkoxy; C₆-C₁₆ aryloxy, C₆-C₆ thioaryloxy, heteroaryloxy including 5-20 atoms, or thioheteroaryloxy including 5-20 atoms, each of which is optionally substituted with 1-5 R^{a}; C₂-C₁₂ alkenyloxy or C₂-C₁₂ alkynyloxy, each of which is optionally substituted with 1-5 R^{e}; C₃-C₁₆ cycloalkyloxy, C₃-C₁₆ cycloalkenyloxy, heterocyclyloxy including 3-16 atoms, heterocycloalkenyloxy including 3-16 atoms, C₇-C₂₀ aralkoxy, or heteroaralkoxy including 6-20 atoms, each of which is optionally substituted with 1-5 R^{f}; mercapto; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or
(iii) C₁-C₁₂ alkyl or C₁-C₁₂ haloalkyl; each of which is optionally substituted with from 1-5 R^{d}; or
(iv) C₂-C₂₀ alkenyl or C₂-C₂₀ alkynyl, each of which is optionally substituted with from 1-10 R^{e};
R⁵ and R⁶ are each, independently, hydrogen, halo, or C₁-C₁₂ alkyl;
R^{a} at each occurrence is, independently:
(i) halo; NR^{b}R^{c}; nitro; azido; hydroxy; C₁-C₂₀ alkoxy or C₁-C₂₀ haloalkoxy, each of which is optionally substituted with from 1-10 R^{d}; C₆-C₁₈ aryloxy or heteroaryloxy including 5-16 atoms, each of which is optionally substituted with from 1-10 R^{a} or R^{a′}; C₂-C₁₂ alkenyloxy or C₂-C₁₂ alkynyloxy, each of which is optionally substituted with 1-5 R^{e}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{f}; mercapto; C₁-C₂₀ thioalkoxy or C₁-C₂₀ thiohaloalkoxy, each of which is optionally substituted with from 1-10 R^{d}; C₆-C₁₈ thioaryloxy or thioheteroaryloxy including 5-16 atoms, each of which is optionally substituted with from 1-10 R^{a} or R^{a′}; C₂-C₁₂ thioalkenyloxy or C₂-C₁₂ thioalkynyloxy, each of which is optionally substituted with 1-5 R^{e}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{f}; cyano; —C(O)R^{g}, —C(O)OR^{g}; —OC(O)R^{g}; —C(O)SR^{g}; —SC(O)R^{g}; —C(S)SR^{g}; —SC(S)R^{g}; C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or
(ii) C₁-C₂₀ alkyl or C₁-C₂₀ haloalkyl, each of which is optionally substituted with from 1-10 R^{d}; or
(iii) C₂-C₂₀ alkenyl or C₂-C₂₀ alkynyl, each of which is optionally substituted with from 1-10 R^{e}; or
(iv) C₃-C₂₀ cycloalkyl C₃-C₂₀ cycloalkenyl, heterocyclyl including 3-20 atoms, or heterocycloalkenyl including 3-20 atoms, C₇-C₂₀ aralkyl or heteroaralkyl including 6-20 atoms, each of which is optionally substituted with from 1-10 R^{f}; or
(v) C₆-C₁₈ aryl or heteroaryl including 5-16 atoms, each of which is optionally substituted with from 1-10 R^{a} or R^{a′};
R^{a′} at each occurrence is, independently, halo; NR^{b}R^{c}; nitro; azido; hydroxy; C₁-C₂₀ alkyl, C₁-C₂₀ haloalkyl, C₂-C₂₀ alkenyl; C₂-C₂₀ alkynyl; C₃-C₂₀ cycloalkyl; C₃-C₂₀ cycloalkenyl, heterocyclyl including 3-20 atoms; heterocycloalkenyl including 3-20 atoms; C₇-C₂₀ aralkyl; heteroaralkyl including 6-20 atoms; C₁-C₂₀ alkoxy; C₁-C₂₀ haloalkoxy; C₆-C₁₈ aryloxy; heteroaryloxy; C₂-C₁₂ alkenyloxy; C₂-C₁₂ alkynyloxy; C₇-C₂₀ aralkoxy; heteroaralkoxy including 6-20 atoms; C₃-C₁₆ cycloalkoxy; C₃-C₂₀ cycloalkenyloxy; heterocyclyloxy including 3-20 atoms; heterocycloalkenyloxy including 3-20 atoms; mercapto; C₁-C₂₀ thioalkoxy; C₁-C₂₀ thiohaloalkoxy; C₆-C₁₈ thioaryloxy; thioheteroaryloxy including 5-16 atoms; C₂-C₁₂ thioalkenyloxy; C₂-C₁₂ thioalkynyloxy; C₇-C₂₀ thioaralkoxy, thioheteroaralkoxy including 6-20 atoms, C₃-C₁₆ thiocycloalkoxy C₃-C₂₀ thiocycloalkenyloxy, thioheterocyclyloxy including 3-20 atoms, or thioheterocycloalkenyloxy including 3-20 atoms, each of which is optionally substituted with from 1-10 R^{f}; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; NR^{h}S(O)ₙR^{j}; or —P(O)(OR^{b})(OR^{c});
each of R^{b}, R^{c}, R^{g}, R^{h}, and Rⁱ, at each occurrence is, independently:
(i) hydrogen; or
(ii) C₁-C₂₀ alkyl or C₁-C₂₀ haloalkyl, each of which is optionally substituted with from 1-10 R^{d}; or
(iii) C₂-C₂₀ alkenyl or C₂-C₂₀ alkynyl, each of which is optionally substituted with from 1-10 R^{e}; or
(iv) C₃-C₂₀ cycloalkyl, C₃-C₂₀ cycloalkenyl, heterocyclyl including 3-20 atoms, or heterocycloalkenyl including 3-20 atoms, C₇-C₂₀ aralkyl, or heteroaralkyl including 6-20 atoms, each of which is optionally substituted with from 1-10 R^{f}; or
(v) C₆-C₁₈ aryl or heteroaryl including 5-16 atoms, each of which is optionally substituted with from 1-10 R^{a}; or
(vi) —C(O)R^{g}, —C(O)OR^{g}; or —S(O)ₙR^{j};
R^{d} at each occurrence is, independently:
(i) NR^{b}R^{c}; nitro; azido; hydroxy; oxo; thioxo; ═NRⁱ; C₁-C₂₀ alkoxy; C₁-C₂₀ haloalkoxy; C₆-C₁₈ aryloxy or heteroaryloxy including 5-16 atoms, each of which is optionally substituted with from 1-10 R^{a}; C₂-C₁₂ alkenyloxy or C₂-C₁₂ alkynyloxy, each of which is optionally substituted with 1-5 R^{e}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{f}; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{a}; C₂-C₁₂ thioalkenyloxy or C₂-C₁₂ thioalkynyloxy, each of which is optionally substituted with 1-5 R^{e}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{f}; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or
(ii) C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{f};
R^{e} at each occurrence is, independently:
(i) halo; NR^{g}R^{h}; nitro; azido; hydroxy; oxo; thioxo; ═NRⁱ; C₁-C₂₀ alkoxy or C₁-C₂₀ haloalkoxy, each of which is optionally substituted with from 1-10 R d; C₆-C₁₈ aryloxy or heteroaryloxy including 5-16 atoms, each of which is optionally substituted with from 1-10 R^{a}; C₂-C₁₂ alkenyloxy or C₂-C₁₂ alkynyloxy, each of which is optionally substituted with 1-5 R^{e}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{f}; mercapto; C₁-C₂₀ thioalkoxy or C₁-C₂₀ thiohaloalkoxy, each of which is optionally substituted with from 1-10 R^{d}; C₆-C₁₈ thioaryloxy or thioheteroaryloxy including 5-16 atoms, each of which is optionally substituted with from 1-10 R^{a}; C₂-Cl₂ thioalkenyloxy or C₂-C₁₂ thioalkynyloxy, each of which is optionally substituted with 1-5 R^{e}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{f}; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or
(ii) C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{f}; or
(iii) C₆-C₁₈ aryl or heteroaryl including 5-16 atoms, each of which is optionally substituted with from 1-10 R^{a};
R^{f} at each occurrence is, independently:
(i) halo; NR^{b}R^{c}; nitro; azido; hydroxy; oxo; thioxo; ═NRⁱ; C₁-C₂₀ alkoxy or C₁-C₂₀ haloalkoxy, each of which is optionally substituted with from 1-10 R^{d}; C₆-C₁₈ aryloxy or heteroaryloxy including 5-16 atoms, each of which is optionally substituted with from 1-10 R^{a} or R^{a′}; C₂-C₁₂ alkenyloxy or C₂-C₁₂ alkynyloxy, each of which is optionally substituted with 1-5 R^{e}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{f}; mercapto; C₁-C₂₀ thioalkoxy or C₁-C₂₀ thiohaloalkoxy, each of which is optionally substituted with from 1-10 R^{d}; C₆-C₁₈ thioaryloxy or thioheteroaryloxy including 5-16 atoms, each of which is optionally substituted with from 1-10 R^{a} or R^{a′}; C₂-C₁₂ thioalkenyloxy or C₂-C₁₂ thioalkynyloxy, each of which is optionally substituted with 1-5 R^{e}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{f}; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or
(ii) C₁-C₂₀ alkyl or C₁-C₂₀ haloalkyl, each of which is optionally substituted with from 1-10 R^{d}; or
(iii) C₂-C₂₀ alkenyl or C₂-C₂₀ alkynyl, each of which is optionally substituted with from 1-10 R^{e}; or
(iv) C₆-C₁₈ aryl or heteroaryl including 5-16 atoms, each of which is optionally substituted with from 1-10 R^{a};
R^{j} is R^{g}, OR^{g}, or NR^{b}R^{c}; and
n is 0, 1 or 2; or a pharmaceutically acceptable salt thereof.

2. The compound of claim 1, wherein R¹is C₆-C₁₀ aryl, optionally substituted with 1-3 R^{a}.

3. The compound of claim 1, wherein R¹is phenyl.

4. The compound of claim 1, wherein R¹has formula (III): 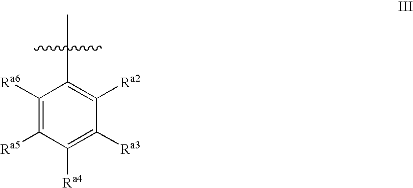
wherein one of R^{a2}, R^{a3}, R^{a4}, R^{a5}, and R^{a6} is:
(i) halo; NR^{b}R^{c}; nitro; hydroxy; C₁-C₁₂ alkoxy or C₁-C₁₂ haloalkoxy, each of which is optionally substituted with from 1-10 R^{d}; C₆-C₁₀ aryloxy or heteroaryloxy including 5-10 atoms, each of which is optionally substituted with from 1-5 R^{a} or R^{a′}; C₂-C₁₂ alkenyloxy or C₂-C₁₂ alkynyloxy, each of which is optionally substituted with 1-5 R^{e}; C₇-C₁₂ aralkoxy, heteroaralkoxy including 6-12 atoms, C₃-C₈ cycloalkoxy, C₃-C₈ cycloalkenyloxy, heterocyclyloxy including 3-8 atoms, or heterocycloalkenyloxy including 3-8 atoms, each of which is optionally substituted with from 1-10 R^{f}; mercapto; C₁-C,₂ thioalkoxy or C₁-C₁₂ thiohaloalkoxy, each of which is optionally substituted with from 1-5 R^{d}; C₆-C₁₈ thioaryloxy or thioheteroaryloxy including 5-10 atoms, each of which is optionally substituted with from 1-5 R^{a} or R^{a′}; C₂-C₁₂ thioalkenyloxy or C₂-C₁₂ thioalkynyloxy, each of which is optionally substituted with 1-5 R^{e}; C₇-C₁₂ thioaralkoxy, thioheteroaralkoxy including 6-12 atoms, C₃-C₈ thiocycloalkoxy, C₃-C₈ thiocycloalkenyloxy, thioheterocyclyloxy including 3-8 atoms, or thioheterocycloalkenyloxy including 3-8 atoms, each of which is optionally substituted with from 1-10 R^{f}; cyano; —C(O)R^{g}, —C(O)OR^{g}; —OC(O)R^{g}; —C(O)SR^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; or —NR^{h}S(O)ₙR^{j}; or
(ii) C₁-C₁₂ alkyl or C₁-C₁₂ haloalkyl, each of which is optionally substituted with from 1-5 R^{d}; or
(iii) C₂-C₁₂ alkenyl or C₂-C₁₂ alkynyl, each of which is optionally substituted with from 1-5 R^{e}; or
(iv) C₇-C₂₀ aralkyl, optionally substituted with from 1-10 R^{f}; and
the others are hydrogen.

5. The compound of claim 4, wherein R^{a3} is nitro.

6. The compound of claim 4, wherein R^{a3} is NR^{b}R^{c}.

7. The compound of claim 6, wherein one of R^{b} and R^{c} is hydrogen, and the other is:
(ii) C₁-C₂₀ alkyl or C₁-C₂₀ haloalkyl, each of which is optionally substituted with from 1-10 R^{d}; or
(iii) C₂-C₂₀ alkenyl or C₂-C₂₀ alkynyl, each of which is optionally substituted with from 1-10 R^{e}; or
(iv) C₃-C₂₀ cycloalkyl, C₃-C₂₀ cycloalkenyl, heterocyclyl including 3-20 atoms, or heterocycloalkenyl including 3-20 atoms, C₇-C₂₀ aralkyl, or heteroaralkyl including 6-20 atoms, each of which is optionally substituted with from 1-10 R^{f}; or
(v) C₆-C₁8 aryl or heteroaryl including 5-16 atoms, each of which is optionally substituted with from 1-10 R^{a}; or
(vi) —S(O)ₙR^{j}, wherein R^{j} is NR^{b}R^{c}.

8. The compound of claim 6, wherein one of R^{b} and R^{c} is hydrogen, and the other is C₃-C₂₀ cycloalkyl, C₃-C₂₀ cycloalkenyl, heterocyclyl including 3-20 atoms, or heterocycloalkenyl including 3-20 atoms, C₇-C₂₀ aralkyl, or heteroaralkyl including 6-20 atoms.

9. The compound of claim 6, wherein one of R^{b} and R^{c} is hydrogen, and the other is C₃-C₁₀ cycloalkyl or heterocyclyl including 3-10 atoms, each of which is optionally substituted with from 1-10 R^{f}.

10. The compound of claim 6, wherein one of R^{b} and R^{c} is hydrogen, and the other is cyclohexyl, optionally substituted with from 1-5 R^{f}.

11. The compound of claim 6, wherein one of R^{b} and R^{c} is hydrogen, and the other is 4-piperidyl, optionally substituted with from 1-2 R^{f}.

12. The compound of claim 6, wherein R^{b} and R^{c} are each independently:
(ii) C₁-C₂₀ alkyl or C₁-C₂₀ haloalkyl, each of which is optionally substituted with from 1-10 R^{d}; or
(iii) C₂-C₂₀ alkenyl or C₂-C₂₀ alkynyl, each of which is optionally substituted with from 1-10 R^{e}; or
(iv) C₃-C₂₀ cycloalkyl, C₃-C₂₀ cycloalkenyl, heterocyclyl including 3-20 atoms, or heterocycloalkenyl including 3-20 atoms, C₇-C₂₀ aralkyl, or heteroaralkyl including 6-20 atoms, each of which is optionally substituted with from 1-10 R^{f}; or
(v) C₆-C₁₈ aryl or heteroaryl including 5-16 atoms, each of which is optionally substituted with from 1-10 R^{a}; or
(vi) —S(O)ₙR^{j}, wherein R^{j} is NR^{b}R^{c}.

13. The compound of claim 6, wherein one of R^{b} and R^{c} is C₃-C₂₀ cycloalkyl, C₃-C₂₀ cycloalkenyl, heterocyclyl including 3-20 atoms, or heterocycloalkenyl including 3-20 atoms, C₇-C₂₀ aralkyl, or heteroaralkyl including 6-20 atoms, each of which is optionally substituted with from 1-10 R^{f}, and the other is —S(O)ₙR^{j}, wherein R^{j} is NR^{b}R^{c}.

14. The compound of claim 6, wherein one of R^{b} and R^{c} is C₃-C₂₀ cycloalkyl, optionally substituted with from 1-10 R^{f}, and the other is —S(O)ₙR^{j}, wherein R^{j} is NR^{b}R^{c}.

15. The compound of claim 6, wherein one of R^{b} and R^{c} is cyclohexyl, optionally substituted with from 1-5 R^{f}, and the other is —S(O)₂NH₂.

16. The compound of claim 1, wherein R⁵ and R⁶ are each, independently, hydrogen, fluoro, or C₁-C₄ alkyl.

17. The compound of claim 1, wherein R⁵ and R⁶ are both hydrogen.

18. The compound of claim 1, wherein one of R⁵ and R⁶ is hydrogen, and the other is C₁-C₄ alkyl.

19. The compound of claim 1, wherein R² and R⁴ are each, independently:
(i) hydrogen; or
(ii) halo; NR^{b}R^{c}; nitro; azido; hydroxy; C₁-C₁₂ alkoxy or C₁-C₁₂ thioalkoxy, each of which is optionally substituted with 1-5 R^{d}; C₁-C₁₂ haloalkoxy; C₆-C₁₆ aryloxy, C₆-C₁₆ thioaryloxy, heteroaryloxy including 5-20 atoms, or thioheteroaryloxy including 5-20 atoms, each of which is optionally substituted with 1-5 R^{a}; C₂-C₁₂ alkenyloxy or C₂-C₁₂ alkynyloxy, each of which is optionally substituted with 1-5 R^{e}; C₃-C₁₆ cycloalkyloxy, C₃-C₁₆ cycloalkenyloxy, heterocyclyloxy including 3-16 atoms, heterocycloalkenyloxy including 3-16 atoms, C₇-C₂₀ aralkoxy, or heteroaralkoxy including 6-20 atoms, each of which is optionally substituted with 1-5 R^{f}; mercapto; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or
(iii) C₁-C₁₂ alkyl or C₁-C₁₂ haloalkyl; each of which is optionally substituted with from 1-5 R^{d}.

20. The compound of claim 1, wherein R² and R⁴ are each, independently, hydrogen; halo; cyano, —C(O)OR^{g}; —C(O)NR^{b}R^{c}; or C₁-C₁₂ alkyl or C₁-C,₂ haloalkyl, each of which is optionally substituted with from 1-5 R^{d}.

21. The compound of claim 1, wherein R² is hydrogen; halo; C₁-C₄ alkyl; or C₁-C₄ haloalkyl.

22. The compound of claim 1, wherein R⁴ is hydrogen; halo; cyano; —C(O)OR^{g}; —C(O)NR^{b}R^{c}; C₁-C₄ haloalkyl; or C₁-C₄ alkyl, optionally substituted with from 1-3 R^{d}.

23. The compound of claim 1, wherein R² and R⁴ are both halo.

24. The compound of claim 1, wherein R² and R⁴ are both hydrogen.

25. The compound of claim 1, wherein one of R² and R⁴ is hydrogen, and the other is:
(ii) halo; NR^{b}R^{c}; nitro; azido; hydroxy; C₁-C₁₂ alkoxy or C₁-C₁₂ thioalkoxy, each of which is optionally substituted with 1-5 R^{d}; C₁-C₁₂ haloalkoxy; C₆-C₁₆ aryloxy, C₆-C₁₆ thioaryloxy, heteroaryloxy including 5-20 atoms, or thioheteroaryloxy including 5-20 atoms, each of which is optionally substituted with 1-5 R^{a}; C₂-C₁₂ alkenyloxy or C₂-C₁₂ alkynyloxy, each of which is optionally substituted with 1-5 R^{e}; C₃-C₁₆-cycloalkyloxy, C₃-C₁₆ cycloalkenyloxy, heterocyclyloxy including 3-16 atoms, heterocycloalkenyloxy including 3-16 atoms, C₇-C₂₀ aralkoxy, or heteroaralkoxy including 6-20 atoms, each of which is optionally substituted with 1-5 R^{f}; mercapto; cyano; —C(O)R^{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or
(iii) C₁-C₁₂ alkyl or C₁-C₁₂ haloalkyl; each of which is optionally substituted with from 1-5 R^{d}; or
(iv) C₂-C₂₀ alkenyl or C₂-C₂₀ alkynyl, each of which is optionally substituted with from 1-10 R^{e}.

26. The compound of claim 1, wherein one of R² and R⁴ is hydrogen, and the other is halo; cyano, —C(O)OR^{g}; —C(O)NR^{b}R^{c}; or C₁-C₁₂ alkyl or C₁-C₁₂ haloalkyl, each of which is optionally substituted with from 1-5 R^{d}.

27. The compound of claim 1, wherein R² is:
(ii) halo; NR^{b}R^{c}; nitro; azido; hydroxy; C₁-C₁₂ alkoxy or C₁-C₁₂ thioalkoxy, each of which is optionally substituted with 1-5 R^{; C}₁-C₁₂ haloalkoxy; C₆-C₁₆ aryloxy, C₆-C₁₆ thioaryloxy, heteroaryloxy including 5-20 atoms, or thioheteroaryloxy including 5-20 atoms, each of which is optionally substituted with 1-5 R^{a}; C₂-C₁₂ alkenyloxy or C₂-C₁₂ alkynyloxy, each of which is optionally substituted with 1-5 R^{e}; C₃-C₁₆ cycloalkyloxy, C₃-C₁₆ cycloalkenyloxy, heterocyclyloxy including 3-16 atoms, heterocycloalkenyloxy including 3-16 atoms, C₇-C₂₀ aralkoxy, or heteroaralkoxy including 6-20 atoms, each of which is optionally substituted with 1-5 R^{f}; mercapto; cyano; —C(O)R_{g}, —C(O)OR^{g}; —OC(O)R^{g}; —C(O)SR^{g}; —SC(O)R^{g}; —C(S)SR^{g}; —SC(S)R^{g}; —C(O)NR^{b}R^{c}; —NR^{h}C(O)R^{g}; —C(NRⁱ)R^{g}; —OC(O)NR^{b}R^{c}; —NR^{h}C(O)NR^{b}R^{c}; —NR^{h}C(O)OR^{g}; —S(O)ₙR^{j}; —NR^{h}S(O)ₙR^{j}; or —P(O)(OR^{b})(OR^{c}); or
(iii) C₁-C₁₂ alkyl or C₁-C₁₂ haloalkyl; each of which is optionally substituted with from 1-5 R¹; or
(iv) C₂-C₂₀ alkenyl or C₂-C₂₀ alkynyl, each of which is optionally substituted with from 1-10 R^{e}; and
R⁴ is hydrogen.

28. The compound of claim 1, wherein R is halo; cyano; —C(O)OR^{g}; —C(O)NR^{b}R^{c}; or C₁-C₁₂ alkyl or C₁-C₁₂ haloalkyl, each of which is optionally substituted with from 1-5 R^{d}, and R⁴ is hydrogen.

29. The compound of claim 1, wherein R² is C₁-C₄ alkyl, and R⁴ is hydrogen.

30. The compound of claim 1, wherein R² is halo, and R⁴ is hydrogen.

31. The compound of claim 1, wherein the compound is selected from the group consisting of:
5-(5-phenyl-3-thienyl)-1,2,5-thiadiazolidin-3-one 1,1-dioxide;
5-(4-chloro-5-phenyl-3-thienyl)-1,2,5-thiadiazolidin-3-one 1,1-dioxide;
5-(2,4-dichloro-5-phenyl-3-thienyl)-1,2,5-thiadiazolidin-3-one 1,1-dioxide;
5-(4-methyl-5-phenyl-3-thienyl)-1,2,5-thiadiazolidin-3-one 1,1-dioxide;
5-[4-methyl-5-(3-nitrophenyl)-3-thienyl] -1,2,5-thiadiazolidin-3-one 1,1-dioxide;
4-methyl-5-(5-phenyl-3-thienyl)-1,2,5-thiadiazolidin-3-one 1,1-dioxide;
5-(4-methyl-5-{3-[(3,3,5,6,-tetramethylcyclohexyl)amino]phenyl}-3-thienyl]-1,2,5-thiadiazolidin-3-one 1,1-dioxide;
5-{5-[3-(cyclohexylamino)phenyl]-3-thienyl }-1,2,5-thiadiazolidin-3-one 1,1-dioxide;
5-[5-(3-{[1-(benzylsulfonyl)piperidine-4-yl]amino}phenyl)-4-methyl-3-thienyl]-thiadiazolidin-3-one 1,1-dioxide;
N-{3-[3-chloro-4-(1,1-dioxido-4-oxo-1,2,5-thiadiazolidin-2-yl)-2-thienyl]phenyl}-N-cyclohexylsulfamide;
5-{4-chloro-5-[3-(cyclohexylamino)phenyl]-3-thienyl}-1,2,5-thiadiazolidin-3-one 1,1-dioxide; and
5-(4-bromo-5-phenyl-3-thienyl)-1,2,5-thiadiazolidin-3-one 1,1-dioxide,
or a pharmaceutically acceptable salt thereof.

32. A pharmaceutical composition comprising a compound of claim 1, or a pharmaceutically acceptable salt thereof, and a pharmaceutically acceptable carrier.

33. A method of treating type 2 diabetes, the method comprising administering to a subject in need thereof an effective amount of a compound of claim 1.

34. A method of treating obesity, the method comprising administering to a subject in need thereof an effective amount of a compound of claim 1.

35. A method of increasing insulin sensitivity, the method comprising administering to a subject in need thereof an effective amount of a compound of claim 1.

36. A method of treating metabolic disorders, the method comprising administering to a subject in need thereof an effective amount of a compound of claim 1.
